(19) United States
(12) Patent Application Publication
(10) Pub. No.: US 2003/0191144 A1
(43) Pub. Date: Oct. 9, 2003

(54) Compounds, compositions and methods for modulating beta-amyloid production

(75) Inventors: Bruce P. Connop, Vancouver (CA); Amelia Grant, Vancouver (CA); David MacDonald, Surrey (CA); Parimal S. Nathwani, Burnaby (CA); Peter B. Reiner, Vancouver (CA); Zaihui Zhang, Richmond (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Active Pass Pharmaceuticals, Inc., Vancouver (CA)

(21) Appl. No.: 10/325,667

(22) Filed: Dec. 19, 2002

Related U.S. Application Data
(63) Continuation-in-part of application No. 10/170,224, filed on Jun. 12, 2002.
(60) Provisional application No. 60/309,257, filed on Jul. 31, 2001.
(60) Provisional application No. 60/297,845, filed on Jun. 12, 2001.

(51) Int. Cl. A61K 31/513

(52) U.S. Cl. 514/269

(57) ABSTRACT

Methods and compositions useful in the treatment of amyloidosis and conditions and diseases associated therewith, such as Alzheimer's disease, are provided. The methods involve administering to a subject in need thereof a pharmaceutical composition including one or more agents that modulate PPARα and/or PPARδ activity, resulting in an inhibition of β-amyloid production and/or release from cells of the subject, particularly brain cells.

### BACKGROUND OF THE INVENTION

1. Field of the Invention

The invention relates to compounds, compositions and methods for regulating the production and/or release of β-amyloid in cells, and provides for alleviation and prevention of β-amyloid production, release and/or plaque development such as occurs in, e.g., Alzheimer's disease.

2. Description of the Related Art

Alzheimer's disease (AD) is a common brain disorder of the elderly and is associated with progressive dementia. The key features of the disease include progressive memory impairment, loss of language and visuospatial skills, and behavior deficits. These changes in cognitive function are the result of degeneration of neurons in the cerebral cortex, hippocampus, basal forebrain, and other regions of the brain. Neuropathological analyses of postmortem Alzheimer's diseased brains consistently reveal the presence of large numbers of neurofibrillary tangles in degenerated neurons and neuritic plaques in the extracellular space and in the walls of the cerebral microvasculature. The neurofibrillary tangles are composed of bundles of paired helical filaments containing hyperphosphorylated tau protein (Lee, V. M and Trojanowski, J. Q. Curr. Opin. Neurobiol. 2:653, 1992). The neuritic plaques consist of deposits of proteinaceous material surrounding a β-amyloid core (Selkoe, D. J., Annu. Rev. Neurosci. 17:489-517, 1994).

Evidence suggests that deposition of amyloid-β peptide (Aβ) plays a significant role in the etiology of Alzheimer's disease. A portion of this evidence is based upon studies that have been generated from data with regard to familial Alzheimer's disease. To date, this aggressive form of Alzheimer's disease has been shown to be caused by missense mutations in (at least) three genes: the β-amyloid precursor protein (APP) gene itself (Goate, A. et al., Nature 349:704-706,1991; Mullan, M. et al., Nature Genet. 1:345-347,1992), and two genes termed presenilins 1 and 2 (Sherrington, R. et al., Nature 375:754-760,1995; Rogaev, E.I. et al., Nature 376:775-778, 1995).

The missense mutations in APP are located in the region of the protein where proteolytic cleavage normally occurs, and expression of these mutants results in increased production of Aβ (Citron, M. et al., Nature 360:672-674,1992, Cai, X-D. et al., Science 259:514-516 1993 and Reaume, A. G. et al., J. Biol. Chem. 271:23380-23388, 1996). Analysis of over 75 mutations of the presenilin genes consistently reveals that these mutations which invariably lead to Alzheimer's disease also result in increased levels of the longer isoform of Aβ known as Aβ42 (Scheuner, D. et al., Nature Medicine 2:864-870,1996 and Selkoe, Physiological Reviews 81:741-766 (2001)). Thus, increased production of Aβ, and in particular Aβ42 is associated with Alzheimer's disease.

Corroborating evidence has been derived from at least two other sources. First, transgenic mice that express either altered APP and/or presenilin genes exhibit neuritic plaques and age-dependent memory deficits (Games, D. et al., “Alzheimer-type neuropathology in transgenic mice overexpressing V717F β-amyloid precursor protein,” Nature 373:523-525 (1995); Masliah, E. et al., “Comparison of neurodegenerative pathology in transgenic mice overexpressing V717F β-amyloid precursor protein and Alzheimer's disease,” J. Neurosci. 16:5795-5811 (1996); Hsiao, K. et al., “Correlative memory deficits, Aβ elevation, and β-amyloid plaques in transgenic mice,” Science 274:99-103 (1996); Holcomb et al., Nature Medicine 4:97-100 (1998)). The second piece of evidence comes from study of patients suffering from Down's syndrome, who develop β-amyloid plaques and other symptoms of Alzheimer's disease at an early age (Mann, D. M. A. and M. M. Esiri, “The pattern of acquisition of plaques and tangles in the brains of patients under 50 years of age with Down's syndrome,” J. Neurol. Sci. 89:169-179 (1989)). Because the APP gene is found on chromosome 21, it has been hypothesized that the increased gene dosage which results from the extra copy of this chromosome in Down's syndrome accounts for the early appearance of β-amyloid plaques (Kang, J. et al., “The precursor protein of Alzheimer's disease β-amyloid A4 protein resembles a cell-surface receptor,” Nature 325:733-736 (1987); Tanzi, R. E. et al., “Amyloid β protein gene: cDNA, mRNA distribution and genetic linkage near the Alzheimer locus,” Science 235:880-884 (1987)). Taken together with the evidence derived from cases of familial Alzheimer's disease, the current data suggest that genetic alterations that result in an increase in AP production can induce Alzheimer's disease. Accordingly, since Aβ deposition is an early and invariant event in Alzheimer's disease, it is believed that treatment that reduces production of Aβ will be useful in the treatment of this disease.

The principal component of the senile plaque is the 4 kDa β-amyloid peptide (Aβ). Ranging between 39 and 43 amino acids in length, Aβ is formed by endoproteolysis of APP. Alternative splicing generates several different isoforms of APP; in neurons, the predominant isoform is 695 amino acids in length (APP695). As APP traverses the endoplasmic reticulum (ER) and trans-Golgi network (TGN), it becomes N- and O-glycosylated and tyrosine-sulfated. Mature holoprotein can be catabolized in several compartments to produce both non- and β-amyloidogenic APP fragments.

APP is expressed and constitutively catabolized in most cells. The dominant catabolic pathway appears to be cleavage of APP within the Aβ sequence by an enzyme provisionally termed α-secretase, leading to release of a soluble ectodomain fragment known as APPsα. In contrast to this non-amyloidogenic pathway, APP can also be cleaved by enzymes known as β- and γ-secretase at the N- and C-termini of the Aβ, respectively, followed by release of Aβ into the extracellular space. To date, BACE has been identified as β-secretase (Vasser et al., Science 286:735-741,1999) and presenilins have been implicated in γ-secretase activity (De Strooper et al., Nature 391:387-390,1998).

The 39-43 amino acid Aβ peptide is produced by sequential proteolytic cleavage of the β-amyloid precursor protein (APP) by the enzyme(s) β and γ secretases. Although Aβ40 is the predominant form produced, 5-7% of total Aβ exists as Aβ42 (Cappai et al., Int J. Biochem. Cell Biol 31:885-889,1999). The length of the Aβ peptide appears to dramatically alter its biochemical/biophysical properties. Specifically, the additional two amino acids at the C-terminus of Aβ42 are very hydrophobic, presumably increasing the propensity of Aβ42 to aggregate. For example, Jarrett et al. demonstrated that Aβ42 aggregates very rapidly in vitro compared to Aβ40, suggesting that the longer forms of Aβ may be the important pathological proteins that are involved in the initial seeding of the neuritic plaques in AD (Jarrett et al., Biochemistry 32:4693-4697, 1993; Jarrett et al., Ann. NY Acad. Sci. 695:144-148, 1993).

This hypothesis has been further substantiated by the recent analysis of the contributions of specific forms of Aβ in cases of genetic familial forms of AD (FAD). For example, the “London” mutant form of APP (APPV7171) linked to FAD selectively increases the production of AP42/43 forms versus Aβ40 (Suzuki et al., Science 264:1336-1340,1994) while the “Swedish” mutant form of APP (APPK670N/M671 L) increases levels of both Aβ40 and Aβ42/43 (Citron et al., Nature 360:672-674, 1992; Cai et al., Science 259:514-516,1993). Also, it has been observed that FAD-linked mutations in the Presenilin-1 (PSi) or Presenilin-2 (PS2) genes will lead to a selective increase in Aβ42/43 production but not Aβ40 (Borchelt et al., Neuron 17:1005-1013, 1996). This finding was corroborated in transgenic mouse models expressing PS mutants that demonstrate a selective increase in brain Aβ42 (Borchelt et al., Neuron 17:1005-1013,1996; Duff et al., Neurodegeneration 5(4):293-298, 1996). Thus the leading hypothesis regarding the etiology of AD is that an increase in AP42 production and/or release is a causative event in the disease pathology.

In addition to a relationship with coronary disease, a relationship exists between serum cholesterol levels and the incidence and the pathophysiology of AD. Epidemiological studies show that patients with elevated cholesterol levels have an increased risk of AD (Notkola et al., Neuroepidemiology. 17(1):14-20, 1998; Jarvik et al., Neurology. 45(6):1092-6, 1995). In addition to the data which suggests that elevated levels of Aβ are associated with AD, other environmental and genetic risk factors have been identified. The best studied of these is polymorphism of the apolipoprotein E (ApoE) gene: patients homozygous for the ε4 isoform of ApoE (apoE4) have consistently been shown to have an increased risk for AD (Strittmatter et al., Proc Natl Acad Sci USA 90:1977-1981 (1993). Because ApoE is a cholesterol transport protein, several groups have observed a correlation between the risk of developing AD and circulating levels of cholesterol (Mahley. Science. 240:622-630,1998; Saunders et al., Neurology. 43:1467-1472,1993; Corder et al., Science. 261:921-923,1993; Jarvik et al., Annals of the New York Academy of Sciences. 826:128-146,1997). Moreover, cholesterol loading increases the production of Aβ protein (Simons et al., PNAS. 95:6460-6464,1998), while pharmacological reduction of cholesterol with the HMG CoA reductase inhibitor simvastatin decreases levels of both Aβ40 and Aβ42 (Fassbender et al., Proc Natl Acad Sci 98:5856-5861 (2001)) in vitro. Consistent with these data are the results of epidemiological studies which have shown that treatment with certain HMG CoA reductase inhibitors, commonly used to normalize cholesterol levels in humans, reduces the prevalence of AD (Wolozin et al., Arch Neurol 57:1439-1443 (2000); Jick et al., Lancet 356:1627-1631 (2000). Taken together, these data suggest a link between regulation of cholesterol levels and AD.

Collectively the wealth of data derived from 1) the biophysical properties of AP, 2) in vitro studies of various cell lines, 3) in vivo studies of transgenic mice and 4) analysis of humans with FAD mutations—all point to Aβ42 as the key pathogenic protein in AD. Thus, there is a need for treatments which selectively inhibit the production and/or release of Aβ42. Such treatments may prove to be extremely valuable in the treatment of both familial and/or sporadic cases of AD.

### BRIEF SUMMARY OF THE INVENTION

This invention is directed to certain pyrimidine compounds, pharmaceutical compositions containing certain pyrimidine compounds, and the use of certain pyrimidine compounds for regulating the production and/or release of β-amyloid in cells, and for alleviation and prevention of β-amyloid production, release and/or plaque development.

In one aspect, the present invention provides a method for modulating the production and/or release of β-amyloid in a cell, comprising treating said cell with a compound of formula (1)

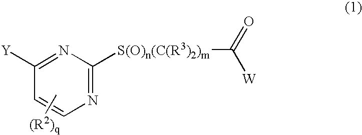

wherein, independently at each occurrence,

W is selected from the group consisting of —OR⁴, —N(R⁵)₂ and —NHN(R⁵)₂;

R² is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;

R³ is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷, —OR⁷, —C(O)OR⁷, —OC(O)R⁷, —C(O)N(R⁶)₂, heterocyclyl and heterocyclylalkyl;

R⁴ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;

R⁵ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;

R⁵ is selected from the group consisting of hydrogen, alkyl, alkenyl, cycloalkyl, cycloalkylalkyl, aralkyl and aryl; and

R⁷ is selected from the group consisting of hydrogen, alkyl and aralkyl;

Y is selected from the group consisting of an aryl radical of 6 to 10 carbon atoms,

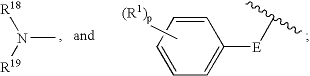

wherein

R¹ is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;

R¹⁸ is hydrogen or lower alkyl radical;

R¹⁹ is hydrogen, H₂N—,

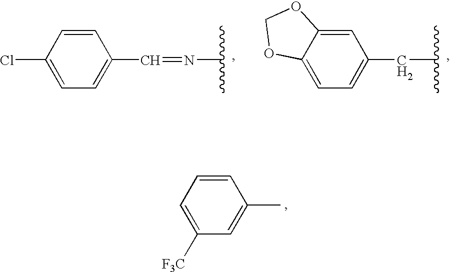

phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl,

m is 0, 1, 2, 3, 4 or 5;

n is 0, 1 or 2;

p is 0, 1,2,3, 4 or 5;

q is 0, 1 or 2;

E is selected from the group consisting of

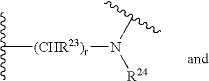

and

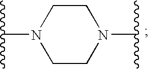

wherein

R²³ is hydrogen or lower alkyl,

R²⁴ is hydrogen or alkyl, and

r is 0, 1, 2 or 3;

as a single stereoisomer, a mixture of stereoisomers, as a racemic mixture of stereoisomers; as a solvate, as a polymorph; or as a pharmaceutically acceptable salt thereof.

In one aspect, the present invention provides a method for modulating the production and/or release of β-amyloid in a cell, comprising treating said cell with a compound of formula (1a)

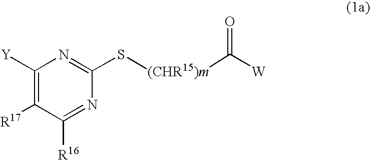

wherein, independently at each occurrence, R¹⁵ and R¹⁷ are each independently selected from the group consisting of hydrogen and lower alkyl radicals; R¹⁶ is selected from the group consisting of hydrogen, halogen and lower alkoxy radicals; R²⁴ is hydrogen or lower alkyl; W is selected from the group consisting of hydroxy, lower alkoxy, —OM and —NHNH₂ radicals, wherein M is selected from the group consisting of alkali metal cation, alkaline earth metal cation and ammonium ion; m is 0, 1, 2 or 3, and Y is as defined in formula (1). Optionally, Y may be selected from the R¹⁸ group consisting of an aryl radical of 6 to 10 carbon atoms,

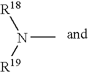

and

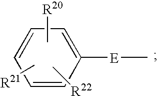

wherein R²⁰ is selected from the group consisting of a lower alkyl radical, a halo radical, an aryl radical of 6 to 10 carbon atoms and a haloaryl radical of 6 to 10 carbon atoms; R²¹ is selected from the group consisting of hydrogen, lower alkyl, lower alkoxy and halo radicals; and R²² is selected from the group consisting of hydrogen and lower alkyl radicals.

In one aspect, the present invention provides a method for modulating the production and/or release of β-amyloid in a cell, comprising treating said cell with a compound of formula (1b)

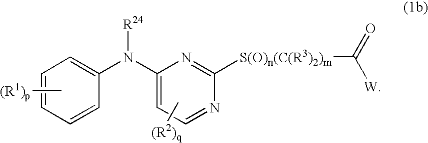

Optionally in compounds of formula (1b), R²⁴ is lower alkyl.

The invention also provides a method of treatment comprising modulating the production and/or release of β-amyloid in a non-human mammal in need of said treatment, said method comprising administering to said non-human mammal a compound of formula (1) that can modulate the production and/or release of β-amyloid in a human, or a composition comprising such a compound.

The invention also provides a method of treatment comprising modulating the production and/or release of β-amyloid in a human in need of said treatment, said method comprising administering to said human a compound of formula (1) that can modulate the production and/or release of β-amyloid in a human, or a composition comprising such a compound.

In addition, the present invention provides compounds of formula (1c)

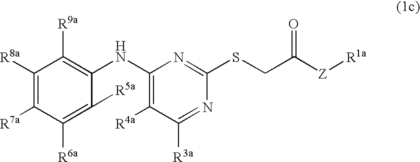

wherein, independently at each occurrence,

R^{1a} is an organic moiety having at least 4 carbons;

Z is selected from —O—, —NH—NH—, and —N(R^{2a})—;

R^{2a} is selected from hydrogen and C₁-C₃₀ organic moieties with the proviso that R^{1a} and R^{2a} can join together with the nitrogen to which they are both attached and form a heterocyclic moiety;

R^{3a} and R^{4a} are each independently selected from the group consisting of hydrogen, halogen, lower alkyl and lower alkoxy radicals;

R^{5a}, R^{6a}, R^{7a}, R^{8a} and R^{9a} are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R^{10a}—N═N—O—R^{11a}, —OR^{12a}, —C(O)OR^{12a}, —N(R^{12a})₂, —C(O)N(R^{12a})₂, —N(R^{12a})C(O)OR^{11a}, heterocyclyl and heterocyclylalkyl;

R^{10a} is a bond or a straight or branched alkylene or alkenylene chain;

R^{11a} is hydrogen, alkyl or aralkyl; and

R^{12a} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl.

In one preferred embodiment, the compounds of formula (1d) exclude the compounds such that Z is not NR^{2a} when R^{3a} is Cl, R^{4a} is H, R^{5a} is H, R^{6a} is H, R^{7a} is H, R^{8a} is methyl and R^{9a} is methyl.

The invention also provides compounds of formula (1d)

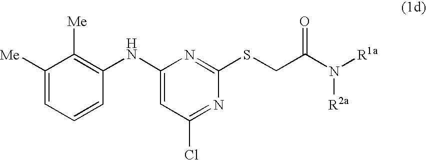

wherein,

R^{1a} is a hydrophobic moiety selected from non-aromatic organic moieties having at least 10 carbon atoms and aromatic moieties having at least 6 carbons, and R^{2a} is hydrogen; or

each of R^{1a} and R^{2a} is selected from hydrophobic organic moieties having at least one carbon atom, with the proviso that R^{1a} and R^{2a} in total have at least six carbon atoms, and with the further proviso that R^{1a} and R^{2a} can join together with the nitrogen to which they are both bonded and form a heterocyclic moiety.

The invention provides a method for modulating the production and/or release of β-amyloid from a cell, comprising treating the cell with an agent, or a composition comprising an agent, that acts as a PPARα and/or PPARδ agonist. In one embodiment, the cell is a brain cell. In a related aspect, the present invention provides a method of inhibiting extracellular β-amyloid levels in the brain of a human in need of such inhibition, comprising administering to the human a pharmaceutical composition comprising an agent that activates PPARα and/or PPARδ activity. In specific embodiments, the β-amyloid is β-amyloid 42.

In another aspect, the present invention provides a method for modulating the production and/or release of β-amyloid in a cell, comprising treating said cell with a compound of the formula (2)

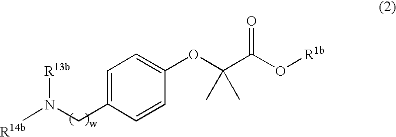

wherein,

R^{1b} is selected from the group consisting of C₁-C₃ alkyl, hydrogen, metal cation and ammonium cation;

R^{13b} and R^{14b} are each independently selected from the group consisting of hydrogen, alkyl, alkenyl, aryl, aralkyl, aralkenyl, cycloalkyl, cycloalkylalkyl, cycloalkylalkenyl, haloalkyl, haloalkenyl, —OR^{12b}, —C(O)OR^{12b}, —N(R^{12b})₂, C(O)N(R^{12b})₂, —N(R^{12b})C(O)OR^{12b}, heterocyclyl and heterocyclylalkyl;

R^{12b} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and

w is 1, 2 or 3.

In a related aspect, the present invention provides a method of treatment comprising modulating the production and/or release of β-amyloid in a human in need of said treatment, said method comprising administering to said human a compound of the formula (2) as defined above and elsewhere herein.

In a related aspect, the present invention provides a method of treatment comprising modulating the production and/or release of β-amyloid in a non-human mammal in need of said treatment, said method comprising administering to said non-human mammal a compound of formula (2) as defined above and elsewhere herein.

The invention also provides a method for modulating the production and/or release of β-amyloid from a cell using an agent selected from the group consisting of (2-pyrimidinylthio) alkanoic acids, esters, amides, hydrazides and 4- and 6-substituted derivatives thereof.

In addition, the invention provides compounds, compositions and methods for regulating the production and/or release of β-amyloid in cells, and provides for alleviation and prevention of β-amyloid production, release and/or plaque development.

The invention yet further provides a method for preferentially reducing production and/or release of Aβ42 relative to one or more other forms of Aβ, in a target that produces and/or releases Aβ42, for instance a target selected from a cell, a human, a non-human mammal, and the brain of a human, comprising administering to the target a compound or pharmaceutical composition comprising a chemical agent as described herein. This method may be used to treat, e.g., a human, wherein said human, e.g., is afflicted with Alzheimer's disease. In another embodiment, said human being treated has a genetic predisposition or environment exposure that increases the likelihood that said person will develop Alzheimer's disease. For example, said human has suffered a head injury and is treated with a compound or composition as described herein. In one embodiment, said human exhibits minimal cognitive impairment suggestive of early stage Alzheimer's disease. In another embodiment, said human has suffered a head injury and is treated with a compound or composition as described herein. Towards this end, the invention also provides a method for delivering to the brain a compound capable of modulating Aβ production and/or release. This delivery system achieves specific delivery of such compounds through conjugating the compounds with a polar lipid or other carrier, achieving effective intracerebral concentration of such compounds efficiently and with specificity.

The invention also provides compounds and compositions useful, for example, in treating Alzheimer's disease wherein the compound, or one or more active agents in the composition, is capable of crossing the blood brain barrier, where such compounds/agents include pirinixic acid in an esterified form, and pirinixic acid conjugated to DHA. In addition, the invention provides a method for delivering to the brain a compound capable of modulating AP production and/or release. This delivery system achieves specific delivery of such compounds through conjugating the compounds with a polar lipid or other carrier, achieving effective intracerebral concentration of such compounds efficiently and with specificity.

The invention further provides compositions of matter comprising a biologically active compound capable of modulating Aβ production and/or release covalently linked to a polar lipid carrier molecule. Preferred embodiments also comprise a spacer molecule having two linker functional groups, wherein the spacer has a first end and a second end and wherein the lipid is attached to the first end of the spacer through a first linker functional group and the biologically active compound is attached to the second end of the spacer through a second linker functional group. In preferred embodiments, the biologically active compound is a PPARα and/or PPARδ agonist. Preferred polar lipids include but are not limited to acyl- and acylated carnitine, sphingosine, ceramide, phosphatidyl choline, phosphatidyl glycerol, phosphatidyl ethanolamine, phosphatidyl inositol, phosphatidyl serine, cardiolipin and phosphatidic acid.

These and other aspects of the present invention will be decribed in detail below.

### BRIEF DESCRIPTION OF THE DRAWINGS

FIG. 1 is a bar graph showing the effect of PPARα and/or PPARδ agonist pirinixic acid on production and/or release of Aβ40 and Aβ42 from SM-4 cells. Cells were treated with 10-500 μM pirinixic acid. After 16 hr, the culture media was harvested and assayed for extracellular levels of Aβ40 and Aβ42 by ELISA. Extracellular Aβ was standardized to propridium iodide fluorescence as a measure of total cell number. Data are expressed as mean±SD with n=3-13 and statistical significance determined by ANOVA with Tukey's post hoc test at ***p<0.001. Double hatched bars indicate Aβ40 levels and hatched bars indicate Aβ42 levels.

FIG. 2 is a bar graph showing the effect of Clofibrate on levels of extracellular levels of Aβ40 and Aβ42 from SM-4 cells. Cells were treated with 10-500 μM Clofibrate. After 16 hrs, the culture media was harvested and assayed for extracellular Aβ40 and Aβ42 by ELISA. Secreted Aβ was standardized to propridium iodide fluorescence as a measure of total cell number. Data are expressed as mean±SD with n=5 and statistical significance determined by ANOVA with Tukey's post hoc test at ***p<0.001. Double hatched bars represent Aβ40 levels as a percent of vehicle, hatched bars represent Aβ42 levels as a percent of vehicle.

FIG. 3 is a bar graph showing the effect of ETYA on levels of extracellular levels of Aβ40 and Aβ42 from SM-4 cells. Cells were treated with 5-100 μM ETYA. After 16 hrs, the culture media was harvested and assayed for extracellular Aβ40 and Aβ42 by ELISA. Secreted Aβ was standardized to propridium iodide fluorescence as a measure of total cell number. Data are expressed as mean±SD with n=5 and statistical significance determined by ANOVA with Tukey's post hoc test at *p<0.05 and **p<0.01. Double hatched bars represent Aβ40 levels as a percent of vehicle, and hatched bars represent Aβ42 levels as a percent of vehicle.

FIG. 4 is a representative micrograph (upper panel) and a bar graph (lower panel) showing the effect of PPARα and/or PPARδ agonist pirinixic acid on cellular APP levels from SM-4 cells. Cells were treated with 50-500 μM pirinixic acid for 16 hours and cellular APP was quantitated by Western blot analysis. Data are expressed as mean±SD with n=4 and statistical significance determined by ANOVA with Tukey's post hoc test at *p<0.05 and **p<0.01.

FIG. 5 is a representative micrograph (upper panel) and a bar graph (lower panel) showing the effect of PPARα and/or PPARδ agonist pirinixic acid on APP_{Sα} release from SM-4 cells. Cells were treated with 50-500 μM pirinixic acid for 16 hours and APP_{Sα} release was quantitated by Western blot analysis. Data are expressed as mean±SD with n=4 and statistical significance determined by ANOVA with Tukey's post hoc test at **p<0.01.

FIG. 6 is a representative micrograph (upper panel) and a bar graph (lower panel) showing the effect of PPARα and/or PPARδ agonist pirinixic acid on C99 levels from SM-4 cells. Cells were treated with 50-500 μM pirinixic acid for 16 hours and C99 was quantitated by Western blot analysis. Data are expressed as mean±SD with n=4 and statistical significance determined by ANOVA with Tukey's post hoc test at **p<0.01.

FIG. 7 is a line graph showing the effect of Compound 1 on secreted Aβ40 and Aβ42 from SM-4 cells. Cells were treated with vehicle (0.01% w/v DMSO) or 50-300 μM Compound 1 for 16 hrs. After the treatment, the culture media was harvested and assayed for Aβ40 and Aβ42 by ELISA. Secreted Aβ was standardized to propridium iodide fluorescence as a measure of total cell number. Data are expressed as mean±SEM with n=4 and statistical significance determined by ANOVA with Tukey's post hoc test at *p<0.05, ***p<0.001

FIG. 8 is a line graph showing the effect of Compound 2 on secreted Aβ40 and Aβ42 in SM-4 cells. Cells were treated with vehicle (0.01% w/v DMSO) or 5-100 μM Compound 2. After 16 hr, the culture media was harvested and assayed for Aβ40 and Aβ42 by ELISA. Secreted Aβ was standardized to propridium iodide fluorescence as a measure of total cell number. Data are expressed as mean±SEM with n=4 and statistical significance determined by ANOVA with Tukey's post hoc test at *p<0.05, ***p<0.001.

FIG. 9 is a bar graph showing the effect of PPARα and/or PPARδ agonist pirinixic acid on secreted Aβ40 and Aβ₄₂ from human neuroblastoma cells. Cells were treated with 100-200 μM of pirinixic acid after transient transfection with Swedish mutant APP. After a 16-hour treatment, the culture media was harvested and assayed for Aβ40 and Aβ42 by ELISA as described in the Methods and Materials. Secreted Aβ was standardized to propridium iodide fluorescence as a measure of total cell number. Data are expressed as mean±SD with n=11 and statistical significance determined by ANOVA with Tukey's post hoc test at ***p<0.001.

FIG. 10 is a bar graph showing the effect of PPARα and/or PPARδ agonist pirinixic acid on Aβtotal and Aβ42 from murine primary cortical neurons infected with APP 695. Cells were treated with 5-250 μM pirinixic acid for 16 hours and Aβtotal and Aβ42 levels were quantitated by immunoprecipitation and ELISA, respectively. Data are expressed as mean±SD with n=6 and statistical significance determined by ANOVA with Tukey's post hoc test at **p<0.01, ***p<0.001.

### DETAILED DESCRIPTION OF THE INVENTION

The invention is based on the inventors' discovery that exposure of mammalian cells to certain PPARα and/or PPARδ agonists modulates, specifically decreases the production and/or release of Aβ, particularly Aβ42, from the cells. Because not all PPARα and/or PPARδ agonists achieve this effect, the invention also provides methods and materials for screening these agonists and related compounds and derivatives to determine their suitability for modulating Aβ production and/or release in vivo. Certain derivatives of the agonists have enhanced ability to penetrate the blood brain barrier.

The invention is also based on the discovery that certain chemical compounds as described below, some of which have previously been shown to decrease cholesterol levels, have an effect on production and/or release of Aβ42.

Definitions

In general, all technical and scientific terms used herein have the same meaning as commonly understood to one of ordinary skill in the art to which this invention belongs, unless clearly indicated otherwise. For clarification, listed below are definitions for certain terms used herein to describe the present invention. These definitions apply to the terms as they are used throughout this specification, unless otherwise clearly indicated.

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art.

By the expression “lower,” used to modify the terms alkyl and alkoxy, applicants mean to limit the aliphatic chain length of those monovalent, branched and unbranched groups of paraffinic derivation to from 1 to 6 carbon atoms. By the term “halo” or “halogens” applicants mean to embrace chlorine, fluorine, iodine and bromine. “Alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In one embodiment, a straight chain or branched chain alkyl has 30 or fewer carbon atoms in its backbone (e.g., C₁-C₃₀ for straight chain, C₃-C₃₀ for branched chain), and more preferably 20 or fewer. Likewise, cycloalkyls have from 4-10 carbon atoms in their ring structure, and more preferably have 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxy alkoxycarbonyloxy, arloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, aralkyl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (benzyl)).

Unless the number of carbons is otherwise specified, “lower alkyl” as used herein means an alkyl group, as defined above, but having from one to ten carbons, more preferably from one to six carbon atoms in its backbone structure. Preferred alkyl groups are lower alkyls having one to three carbon atoms.

“Alkylene chain” refers to a straight or branched divalent hydrocarbon chain consisting solely of carbon and hydrogen, containing no unsaturation and having from one to twenty carbons atoms, preferably having from one to eight carbons, e.g., methylene, ethylene, propylene, n-butylene, and the like.

“Alkenyl” refers to a straight or branched monovalent hydrocarbon chain radical consisting solely of carbon and hydrogen atoms, containing at least one double bond, having from one to twenty carbon atoms, preferably from one to eight carbon atoms, and which is attached to the rest of the molecule by a single bond, e.g., ethenyl, prop-1-enyl, but-1-enyl, pent-1-enyl, penta-1,4-dienyl, and the like.

“Alkynyl” refers to a straight or branched monovalent hydrocarbon chain radical consisting solely of carbon and hydrogen atoms, containing at least one triple bond, having from one to twenty carbon atoms, preferably from one to eight carbon atoms, and which is attached to the rest of the molecule by a single bond, e.g., ethynyl, prop-1-ynyl, pent-1-ynyl, penta-1,4-diynyl, and the like.

“Alkoxy” refers to a radical of the formula —ORₐ where Rₐ is an alkyl radical as defined above, e.g., methoxy, ethoxy, n-propoxy, 1-methylethoxy (iso-propoxy), n-butoxy, n-pentoxy, 1,1-dimethylethoxy (t-butoxy), and the like.

“Aryl” refers to a phenyl or naphthyl radical. Unless stated otherwise specifically in the specification, the term “aryl” or the prefix “ar-” (such as in “aralkyl”) is meant to include phenyl and naphthyl radicals optionally substituted by one or more substituents as described above in connection with the term “alkyl”. In one embodiment of the invention, the aryl group is phenyl. In another or additional embodiment, the aryl group has a single substituent. In another or additional embodiment, the aryl group has two substituents.

In one aspect of the invention, substituted aryl refers to an aryl group substituted with one or more groups selected from alkyl, heteroalkyl, haloalkyl, haloalkoxy, aryl, halo, nitro, cyano, —NHOH, —OR₇, —SR₇, —C(O)OR₇, —OC(O)R₇, —C(O)N(R₆)₂, —C(S)R₆, —C(O)R₆, —N(R₆)₂, —N(R₆)C(O)R₆, —N(R₆)C(O)OR₇, —S(O)ₜR₆ (where t is 0 to 2), —S(O)ₜN(R₆)₂ (where t is 0 to 2), —OC(S)NR₆, —NR₆C(S)OR₇, —NR₆S(O)ₜR₆ (where t is 0 to 2), heterocyclyl, heterocyclylalkyl with R₆ and R₇ as defined above.

“Aryloxy” refers to a radical of the formula —OR_{b} where R_{b} is an aryl radical as defined above, e.g., phenoxy and the like.

“Aralkyl” refers to a radical of the formula —RₐR_{b} where Rₐ is an alkyl radical as defined above and R_{b} is one or more aryl radicals as defined above, e.g., benzyl, diphenylmethyl, and the like. The aryl radical may be optionally substituted as described above.

“Aralkenyl” refers to a radical of the formula —Rₑ—R_{b} where R_{b} is an aryl radical as defined above and Rₑ is an alkenyl radical as defined above, e.g., 2-phenylethenyl, and the like.

“Carboxy” refers to the —C(O)OH radical.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group of substituents identified above in connection with the “alkyl” groups. In one embodiment, the alkyl group is mono-substituted. In another embodiment, the alkyl group is unsubstituted. In another embodiment, “cycloalkyl” refers to radicals which are by one or more substituents independently selected from the group consisting of alkyl, alkoxy, halo, haloalkyl, haloalkoxy, hydroxy, amino, and carboxy.

“Halo” refers to bromo, chloro, iodo or fluoro.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Heteroalkyl” refers to an alkyl radical as defined above substituted with one or more individually selected from halo, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), with R⁶ and R⁷ as defined above and the substitution can occur on any carbon of the alkyl group, e.g., —CH₂CH(CH₃)CH₂NH₂, —CH₂CH₂OH, —CH₂CH(F)CH₂NH₂, and the like.

“Heteroalkenyl” refers to an alkenyl radical as defined above substituted with one or more individually selected from halo, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), with R⁶ and R⁷ as defined above and the substitution can occur on any carbon of the alkenyl group, e.g., —CH═CH(CH₃)CH₂NH₂, —CH═CH₂CH₂OH, and the like.

“Heteroalkynyl” refers to an alkynyl radical as defined above substituted with one or more individually selected from halo, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), with R⁶ and R⁷ as defined above and the substitution can occur on any carbon of the alkynyl group, e.g., —C≡CCH₂NH₂, —C≡CCH₂OH, and the like.

“Heterocyclyl” refers to a stable 3- to 15-membered ring radical which consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be aromatic or partially or fully saturated. The heterocyclyl radical may not be attached to the rest of the molecule at any heteroatom atom. Examples of such heterocyclyl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,5]imidazo[1,2-a]pyridinyl; carbazolyl, cinnolinyl, dioxolanyl, decahydroisoquinolyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Unless stated otherwise specifically in the specification, the term “heterocyclyl” is meant to include heterocyclyl radicals as defined above which are optionally substituted by one or more substituents as defined above in connection with the description of “alkyl” groups. In one embodiment of the invention, the heterocyclic group does not have a substituent. In another embodiment, the heterocyclic group has a single substituents. In one embodiment of the invention, the heterocyclyl group is substituted by one or more substituents selected from the group consisting of alkyl, heteroalkyl, haloalkyl, haloalkoxy, aryl, halo, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl, heterocyclylalkyl with R⁶ and R⁷ as defined above.

“N-heterocyclyl” refers to a heterocyclyl radical as defined above wherein the one to five heteroatoms contained therein are selected only from nitrogen, e.g., pyridinyl, tetrazolyl, pyrazolyl, isoquinolinyl, quinolinyl, and phthalazinyl and the like.

“Heterocyclylalkyl” refers to a radical of the formula —RₐR_{d} where Rₐ is an alkyl radical as defined above and R_{d} is a heterocyclyl radical as defined above, and if the heterocyclyl is a nitrogen-containing heterocyclyl, the heterocyclyl may be attached to the alkyl radical at the nitrogen atom. The heterocyclyl radical may be optionally substituted as defined above.

“Heterocyclylcarbonyl” refers to a radical of the formula —C(O)—R_{d} where R_{d} is a heterocyclyl radical as defined above, and if the heterocyclyl is a nitrogen-containing heterocyclyl, the heterocyclyl may be attached to the carbonyl at the nitrogen atom.

“Hydrocarbon” refers to a compound formed entirely of carbon and hydrogen (including isotopes thereof), while “hydrocarbyl” refers to a hydrocarbon radical.

As used herein, compounds which are “commercially available” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury CN), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified though various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H, O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 5th Ed., Wiley-Interscience, N.Y., 2000. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent.

“Mammal” includes humans and domesticated animals, such as cats, dogs, swine, cattle, sheep, goats, horses, rabbits, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” and “salts thereof” in the compounds of the present invention refers to pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“Pharmaceutically acceptable excipient” as used herein is intended to include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or stabilizer which has been approved by the United States Food and Drug Administration as being acceptable for use in humans or domestic animals.

“Treating” or “treatment” as used herein covers the treatment of a disorder in a mammal, preferably a human, which disorder is characterized by the accumulation or deposition of β-amyloid peptide, and includes:

(i) preventing the disorder from occurring in a mammal, in particular a human, when such mammal is predisposed to the disorder but has not yet been diagnosed as having it;

(ii) inhibiting the disorder, i.e., arresting its development; or

(iii) relieving the disorder, i.e., causing regression of the condition.

Compounds

Pirinixic Acid, and Analogs and Derivatives Thereof

In this invention, compounds of formula (1) are defined as follows:

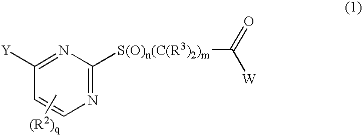

wherein, independently at each occurrence,

W is selected from the group consisting of —OR⁴, —N(R⁵)₂ and —NHN(R⁵)₂;

R² is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;

R³ is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷, —OR⁷, —C(O)OR⁷, —OC(O)R⁷, —C(O)N(R⁶)₂, heterocyclyl and heterocyclylalkyl;

R⁴ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;

R⁵ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;

R⁶ is selected from the group consisting of hydrogen, alkyl, alkenyl, cycloalkyl, cycloalkylalkyl, aralkyl and aryl; and

R⁷ is selected from the group consisting of hydrogen, alkyl and aralkyl;

Y is selected from the group consisting of an aryl radical of 6 to 10 carbon atoms,

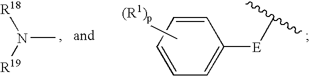

wherein

R¹ is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶) C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;

R¹⁸ is hydrogen or lower alkyl radical;

R¹⁹ is hydrogen, H₂N—,

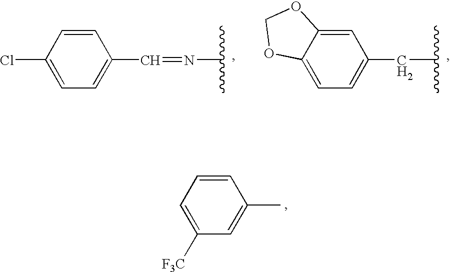

phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy;

m is 0, 1, 2, 3, 4 or 5;

n is 0, 1 or 2;

p is 0, 1, 2, 3, 4 or 5;

q is 0, 1 or 2;

E is selected from the group consisting of

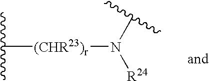

and

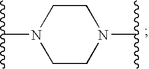

wherein

R²³ is hydrogen or lower alkyl;

R²⁴ is hydrogen or alkyl; and

r is 0, 1, 2 or 3.

The compound(s) of formula (1), as well as the compounds of formulae (1a), (1b), (1c), (1d) and (2) as defined below, may be, for example, a single stereoisomer, a mixture of stereoisomers, a racemic mixture of stereoisomers; in solvated form, as a polymorph; or as a pharmaceutically acceptable salt thereof. In one aspect, the invention provides prodrug forms of compounds of formulae (1), (1a), (1b), (1c), (1d) and (2).

In various aspects of the invention, the compounds of formula (1) are described by formula (1a)

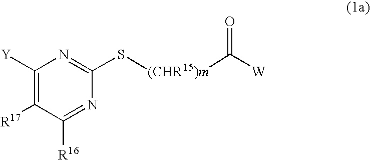

wherein, independently at each occurrence,

R¹⁵ and R¹⁷ are each independently selected from the group consisting of hydrogen and lower alkyl radicals;

R¹⁶ is selected from the group consisting of hydrogen, halogen and lower alkoxy radicals;

R²⁴ is hydrogen or lower alkyl;

W is selected from the group consisting of hydroxy, lower alkoxy, —OM and —NHNH₂ radicals, wherein M is selected from the group consisting of alkali metal cation, alkaline earth metal cation and ammonium ion; and

m is 0, 1, 2 or 3.

Optionally, in compounds of formula (1a), Y is selected from the group consisting of an aryl radical of 6 to 10 carbon atoms,

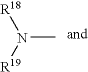

and

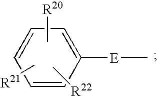

wherein

R²⁰ is selected from the group consisting of a lower alkyl radical, a halo radical, an aryl radical of 6 to 10 carbon atoms and a haloaryl radical of 6 to 10 carbon atoms;

R²¹ is selected from the group consisting of hydrogen, lower alkyl, lower alkoxy and halo radicals; and

R²² is selected from the group consisting of hydrogen and lower alkyl radicals.

The compound of formula (1a) are exemplified by pirinixic acid with the structure:

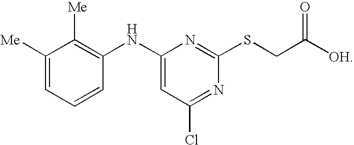

In other aspects of the invention, the compounds of formula (1) are described by formula (1b)

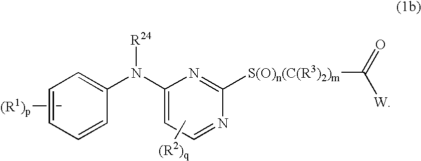

Optionally, in compounds of formula (1b), and independently at each occurrence, W is selected from the group consisting of —OR⁴ and —N(R⁵)₂;

p is 1, 2, 3 or 4;

q is 1 or 2;

m is 1, 2, 3, 4 or 5;

R¹ has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;

R² has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2);

R³ has a formula weight of less than 200 and is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶) C(O)OR⁷, —OC(S)NR⁶, —NR⁶C(S)OR⁷, —OR⁷, —C(O)OR⁷, —OC(O)R⁷, and —C(O)N(R⁶)₂;

R⁴ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;

R⁵ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;

R⁶ has a formula weight of less than 500 and is selected from the group consisting of hydrogen, alkyl, alkenyl, cycloalkyl, cycloalkylalkyl, aralkyl and aryl; and

R⁷ has a formula weight of less than 500 and is selected from the group consisting of hydrogen, alkyl and aralkyl. In a preferred embodiment, R²⁴ is lower alkyl.

In one aspect, the compounds of the invention have the formula

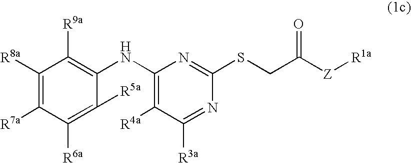

wherein, independently at each occurrence, R^{1a} is hydrogen in one embodiment, while R^{1a} is an organic moiety having at least 1, at least 2, at least 3, at least 4 carbons, and at least 5 carbons in various additional embodiments; Z is selected from —O—, —NH—NH—, and —N(R^{2a})—; R^{2a} is selected from hydrogen and C₁-C₃₀ organic moieties with the proviso that R^{1a} and R^{2a} can join together with the nitrogen to which they are both attached and form a heterocyclic moiety; R^{3a} and R^{4a} are each independently selected from the group consisting of hydrogen, halogen, lower alkyl and lower alkoxy radicals; R^{5a}, R^{6a}, R^{7a}, R^{8a} and R^{9a} are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R^{11a}, —OR^{2a}, C(O)OR^{12a}, —N(R^{12a})₂, —C(O)N(R^{12a})₂, —N(R^{12a})C(O)OR^{11a}, heterocyclyl and heterocyclylalkyl; R^{10a} is a bond or a straight or branched alkylene or alkenylene chain; R^{11a} is hydrogen, alkyl or aralkyl; and R^{12a} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl.

In compounds of formula (1c), one or more of the following criteria may be applied in order to further define the compounds of interest, where any two or more criteria may be combined together so long as no two of the criteria are inconsistent with one another: Z is —O—, Z is —NH—NH—, Z is —N(H)—, Z is —N(R^{2a})—, R^{1a} is an organic group having less than 30 carbons, R^{1a} is an organic group having less than 25 carbons, R^{1a} is an organic group having less than 20 carbons, R^{1a} is an organic group having less than 15 carbons, R^{1a} is an organic group having at least 2 carbons, R^{1a} is an organic group having at least 3 carbons, R^{1a} is an organic group having at least 4 carbons, R^{1a} is an organic group having at least 5 carbons, R^{1a} is an organic group having at least 6 carbons, R^{1a} has a formula weight of less than 1,000; R^{1a} has a formula weight of less than 900, R^{1a} has a formula weight of less than 800, R^{1a} has a formula weight of less than 700, R^{1a} has a formula weight of less than 600, R^{1a} has a formula weight of less than 500, R^{1a} has a formula weight of less than 400, R^{1a} is alkyl, R^{1a} is alkenyl, R^{1a} is aryl, R^{1a} is aralkyl, R^{1a} is aralkenyl, R^{1a} is cycloalkyl, R^{1a} is cycloalkylalkyl, R^{1a} is cycloalkylalkenyl, R^{1a} is halogen, R^{1a} is haloalkyl, R^{1a} is haloalkenyl, R^{1a} is cyano, R^{1a} is nitro, R^{1a} is R^{10a} —N═N—O—R^{11a}, R^{1a} is —OR^{12a}, R^{1a} is —C(O)OR^{12a}, R^{1a} is —N(R^{2a})₂, R^{1a} is —C(O)N(R^{12a})₂, R^{1a} is —N(R^{12a})C(O)OR^{11a}, R^{1a} is heterocyclyl, R^{1a} is heterocyclylalkyl, R^{1a} is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4; R^{1a} is a fragment of insulin wherein said insulin fragment binds to an insulin receptor, e.g., R^{1a} is a fragment of insulin that consists of (a) a peptide chain having 14 to 21 amino acid residues from the N-terminus of insulin chain A; and (b) another peptide chain having 16 to 22 amino acid residues from the N-terminus of insulin chain B; R^{1a} is a protein that binds to a transferrin receptor; R^{1a} is an antibody or a fragment thereof capable of binding to a ligand in the brain, e.g., R^{1a} is a monoclonal antibody; R^{1a} is a growth factor, e.g., EGF; R^{5a} is hydrogen; R^{5a} is halogen; R^{5a} is lower alkyl; R^{5a} is lower alkoxy; R^{6a} is hydrogen; R^{6a} is halogen; R^{6a} is lower alkyl; R^{6a} is lower alkoxy; R^{7a} is hydrogen; R^{7a} is halogen; R^{7a} is lower alkyl; R^{7a} is lower alkoxy; R^{8a} is hydrogen; R^{7a} is halogen; R^{7a} is lower alkyl; R^{8a} is lower alkoxy; R^{9a} is hydrogen; R^{9a} is halogen; R^{9a} is lower alkyl; R^{9a} is lower alkoxy; R^{1a} imparts to the compound the property of enhanced penetration of the blood brain barrier relative to the corresponding compound wherein R^{1a} is hydrogen.

In a preferred embodiment, in describing compound of formula (1c), and independently at each occurrence,

R^{1a} is an organic moiety having at least 4 carbons;

Z is selected from —O—, —NH—NH—, and —N(R^{2a})—;

R^{2a} is selected from hydrogen and C₁-C₃₀ organic moieties with the proviso that R^{1a} and R^{2a} can join together with the nitrogen to which they are both attached and form a heterocyclic moiety;

R^{3a and R}^{4a} are each independently selected from the group consisting of hydrogen, halogen, lower alkyl and lower alkoxy radicals;

R^{5a}, R^{6a}, R^{7a}, R^{8a} and R^{9a} are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R^{10a}—N═N—O—R^{11a}, —OR^{12a}, —C(O)OR^{12a}, —N(R^{12a})₂, —C(O)N(R^{12a})₂, —N(R^{12a})C(O)OR^{11a}, heterocyclyl and heterocyclylalkyl;

R^{10a} is a bond or a straight or branched alkylene or alkenylene chain;

R^{11a} is hydrogen, alkyl or aralkyl; and

R^{12a} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl.

Optionally, these compounds are described with the proviso that Z is not NR^{2a} when R^{3a} is Cl, R^{4a} is H, R^{5a} is H, R^{6a} is H, R^{7a} is H, R^{8a} is methyl and R^{9a} is methyl.

In another aspect, the compound of formula (1) may be described as an amide having the formula (1d)

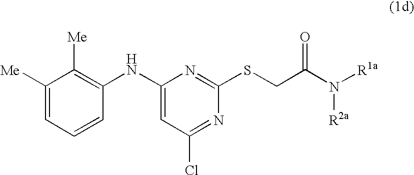

wherein R^{1a} and R^{2a} are hydrogen or organic moieties. In various embodiments of the invention, one, two or more of the following criteria may be further applied to describe compounds of this formula, where any two or more criteria may be combined so long as those criteria are not inconsistent with one another: R^{1a} is aromatic, R^{1a} is non-aromatic, R^{1a} is aliphatic, R^{1a} has no more than 30 carbon atoms, R^{1a} has no more than 25 carbon atoms, R^{1a} has no more than 20 carbon atoms, R^{1a} has at least 2 carbon atoms, R^{1a} has at least 3 carbon atoms, R^{1a} has at least 4 carbon atoms, R^{1a} has at least 5 carbon atoms, R^{1a} has at least 6 carbon atoms, R^{1a} has at least 7 carbon atoms, R^{1a} has at least 8 carbon atoms, R^{1a} has at least 9 carbon atoms, R^{1a} has at least 10 carbon atoms, R^{1a} has a formula weight of less than 1,000; R^{1a} has a formula weight of less than 900, R^{1a} has a formula weight of less than 800, R^{1a} has a formula weight of less than 700, R^{1a} has a formula weight of less than 600, R^{1a} has a formula weight of less than 500, R^{1a} has a formula weight of less than 400, R^{1a} is alkyl, R^{1a} is alkenyl, R^{1a} is aryl, R^{1a} is aralkyl, R^{1a} is aralkenyl, R^{1a} is cycloalkyl, R^{1a} is cycloalkylalkyl, R^{1a} is cycloalkylalkenyl, R^{1a} is halogen, R^{1a} is haloalkyl, R^{1a} is haloalkenyl, R^{1a} is cyano, R^{1a} is nitro, R^{1a} is R^{10a}—N═N—O—R^{11a}, R^{1a} is —OR^{12a}, R^{1a} is —C(O)OR^{12a}, R^{1a} is —N(R^{2a})₂, R^{1a} is —C(O)N(R^{12a})₂, R^{1a} is —N(R^{12a})C(O)OR^{11a}, where R^{10a}, R^{11a} and R^{12a} are defined elsewhere herein, R^{1a} is heterocyclyl, R^{1a} is heterocyclylalkyl, R^{1a} is a hydrocarbon, R^{1a} is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4; R^{1a} is a fragment of insulin wherein said insulin fragment binds to an insulin receptor, e.g., R^{1a} is a fragment of insulin that consists of (a) a peptide chain having 14 to 21 amino acid residues from the N-terminus of insulin chain A; and (b) another peptide chain having 16 to 22 amino acid residues from the N-terminus of insulin chain B; R^{1a} is a protein that binds to a transferrin receptor; R^{1a} is an antibody or a fragment thereof capable of binding to a ligand in the brain, e.g., R^{1a} is a monoclonal antibody; R^{1a} is a growth factor, e.g., EGF; R^{1a} imparts to the compound the property of enhanced penetration of the blood brain barrier relative to the corresponding compound wherein R^{1a} is hydrogen, R^{2a} is hydrogen, R^{2a} is selected from groups that R^{1a} may be as defined above, R^{1a} and R^{2a} can join together with the nitrogen to which they are both bonded and form a heterocyclic moiety, R^{1a} and R^{2a} in total have at least 2, or at least 3, or at least 4, or at least 5, or at least 6 carbons. For example, in one embodiment, R^{1a} is a hydrophobic moiety selected from non-aromatic organic moieties having at least 10 carbon atoms and aromatic moieties having at least 6 carbons, and R^{2a} is hydrogen. As another example, in another embodiment, each of R^{1a} and R^{2a} are selected from hydrophobic organic moieties having at least one carbon atom, with the proviso that R^{1a} and R^{2a} in total have at least six carbon atoms, and with the further proviso that R^{1a} and R^{2a} can join together with the nitrogen to which they are both bonded and form a heterocyclic moiety.

In a preferred embodiment of compounds of formula (1d):

R^{1a} is a hydrophobic moiety selected from non-aromatic organic moieties having at least 10 carbon atoms and aromatic moieties having at least 6 carbons, and R^{2a} is hydrogen; or

each of R^{1a} and R^{2a} is selected from hydrophobic organic moieties having at least one carbon atom, with the proviso that R^{1a} and R^{2a} in total have at least six carbon atoms, and with the further proviso that R^{1a} and R^{2a} can join together with the nitrogen to which they are both bonded and form a heterocyclic moiety.

The terms (R¹)ₚ— and (R²)_{q}— are utilized herein to indicate that a number “p” of R¹ groups are bonded to the carbocyclic aromatic ring of the compound, and a number “q” of R² groups are bonded to the heterocyclic aromatic ring of the compound. When p is zero, then there are no R¹ groups present on the compound, and the carbocyclic aromatic ring is unsubstituted phenyl. Likewise, when q is zero, then there are no R² groups present on the compound. However, when p is greater than zero, then “p” R¹ groups are bonded to the carbon atoms of the carbocyclic aromatic ring of the compound, and likewise when q is greater than zero, then “q” R² groups are bonded to the carbon atoms of the heterocyclic ring of the compound. In each case when an R¹ or R² group is present in the compound, the R¹ and/or R² group replaces a hydrogen atom that would otherwise be bonded to the ring carbon.

Preferred compounds include those of the formula:

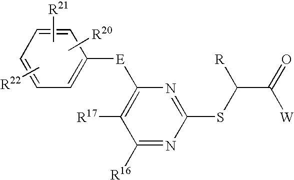

wherein, independently at each occurrence, R¹⁶ is selected from the group consisting of hydrogen and chloro radicals; R, R¹⁷ and R²² are independently selected from the group consisting of hydrogen and lower alkyl radicals; R²⁰ is selected from the group consisting of lower alkyl; lower alkoxy, aryl of 6 to 10 carbon atoms, haloaryl of 6 to 10 carbon atoms and halo radicals; R²¹ is selected from the group consisting of —H, lower alkyl, halo and lower alkoxy radicals; E is selected from the group consisting of

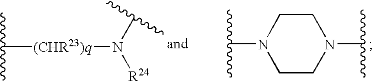

wherein R²³ and R²⁴ are independently —H or lower alkyl and q is an integer from 0 to 3, providing that when q is 0 and R²⁰ is lower alkoxy, R²¹ is lower alkyl, lower alkoxy or halo; and Z is selected from the group consisting of —OH, OM, lower alkoxy and —(NH)_{P}—NH₂, in which p is an integer from 0 to 1 and M is an alkali metal, alkaline earth metal or ammonium cation.

Preferred compounds are the [4-chloro-6-arylamino-2-pyrimidinylthio] acetic acid, alkali metal salt, amide, hydrazide and lower alkyl ester in which the aryl group contains from 7 to 12 carbon atoms, and the 6-para-chlorophenylamino and 6-para-chlorobenzylamino analogues thereof.

More preferred compounds of the invention may be represented by the following formula:

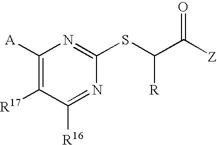

wherein, independently at each occurrence, A is a member selected from the group consisting of aryl of 6 to 10 carbon atoms and

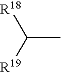

wherein R¹⁸ is —H or lower alkyl and R¹⁹ is hydrogen, H₂N—,

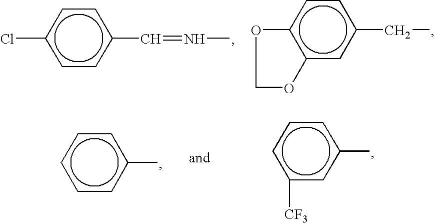

R is selected from the group consisting of —H and lower alkyl; R¹⁷ is selected from the group consisting of —H and lower alkyl; R¹⁶ is selected from the group consisting of —H, chloro and lower alkoxy radicals, with the proviso that when A is the amino or phenylamino group R¹ is chloro or lower alkoxy; and Z is selected from the group consisting of —NHNH₂, lower alkoxy, —OH and OM, wherein M is an alkali metal, alkaline earth metal or ammonium cation.

Specifically preferred compounds include:

(4,6-dichloro-2-pyrimidinylthio)acetic acid, ethyl ester.

(4-amino-6-chloro-2-pyrimidinylthio)acetic acid ethyl ester.

(4-anilino-6-chloro-2-pyrimidinylthio)acetic acid ethyl ester.

(4-chloro-6-(p-chloroanilino)-2-pyrimidinylthio)acetic acid ethyl ester.

[4-chloro-6-(p-fluoroanilino)-2-pyrimidinylthio]acetic acid ethyl ester.

[4-chloro-6-(α,α,α-trifluoro-m-toluidino)-2-pyrimidinylthio]acetic acid ethyl ester.

[4-chloro-6-(2,3-xylidino)-2-pyrimidinylthio]acetic acid ethyl ester.

[4-chloro-6-(2,4,6-trimethylanilino)-2-pyrimidinylthio]acetic acid ethyl ester.

[4-chloro-6-(p-methoxyanilino)-2-pyrimidinylthio]acetic acid ethyl ester.

[4-(4-biphenylylamino)-6-chloro-2-pyrimidinylthio]acetic acid ethyl ester.

(4-chloro-6-[4-(p-chlorophenyl)-1-piperazinyl]-2-pyrimidinylthio)acetic acid ethyl ester.

[4-chloro-6-(2,3-xylindino)-2-pyrimidinylthio]acetic acid.

[4-chloro-6-(2,3-xylindino)-2-pyrimidinylthio]acetamide.

[4-chloro-6-(2,3-xylindino)-2-pyrimidinylthio]acetic acid hydrazide.

[4-chloro-6-(p-chlorobenzylamino)-2-pyrimidinylthio]acetic acid, ethyl ester.

[4-chloro-6-(p-fluorobenzylamino)-2-pyrimidinylthio]acetic acid, ethyl ester.

[4-chloro-6-(3,4-dichlorobenzylamino)-2-pyrimidinylthio]acetic acid, ethyl ester.

[4-chloro-6-(2,4-dimethoxyanilino)-2-pyrimidinylthio]acetic acid.

[4-chloro-6-(2,4-dimethyqlbenzylamino)-2-pyrimidinylthio]acetic acid ethyl ester.

[4-chloro-6-(p-chlorophenethylamino)-2-pyrimidinylthio]acetic acid ethyl ester.

(4-chloro-6-[(p-chlorobenzyl)methylamino]-2-pyrimidinylthio)acetic acid ethyl ester.

[4-chloro-6-(p-chloro-α-methylbenzylamino)-2-pyrimidinylthio]acetic acid.

(4-chloro-6-[3,4-(methylenedioxy)benzylamino]-2-pyrimidinylthio))acetic acid ethyl ester.

[4-chloro-6-(p-chlorobenzylidenehydrazino)-2-pyrimidinylthio]acetic acid ethyl ester.

(4-chloro-6-[(p-fluorobenzylidene)hydrazino]-2-pyrimidinylthio)acetic acid ethyl ester.

(4-chloro-6-hydrazino-2-pyrimidinylthio)acetic acid, ethyl ester, hydrochloride.

[4-chloro-6-(p-chlorobenzylamino)-2-pyrimidinylthio]acetic acid.

(4-chloro-6-(p-chlorobenzylamino)-2-pyrimidinylthio)acetic acid hydrazide.

2-(4,6-dichloro-2-pyrimidinylthio)propionic acid ethyl ester.

2-[4-chloro-6-(p-chlorobenzylamino)-2-pyrimidinylthio]propionic acid.

(4-chloro-6-phenyl-2-pyrimidinylthio)acetic acid ethyl ester.

(4-methoxy-6-phenyl-2-pyrimidinylthio)acetic acid.

[4-(p-chlorobenzylamino)-2-pyrimidinylthio]acetic acid ethyl ester.

[4-(p-chlorobenzyl)methylamino-2-pyrimidinylthio]acetic acid ethyl ester,

(4,6-dichloro-5-methyl-2-pyrimidinylthio)acetic acid, ethyl ester.

[4-chloro-6-(p-chlorobenzylamino)-5-methyl-2-pyrimidinylthio]acetic acid, ethyl ester.

(4-chloro-6-[p-chlorobenzyl)methylamino]-5-methyl-2-pyrimidinylthio)acetic acid, ethyl ester.

[4-chloro-6-2,3-xylidino)-2-pyrimidinylthio]acetic acid, sodium salt, hemihydrate.

PPARα and PPARδ Agonists

As discussed in greater detail below, this invention discloses, for the first time, the use of these compounds and derivatives thereof to decrease β-amyloid production and/or release from cells, specifically the 42-amino acid form, Aβ42, which has been implicated in the development and progression of Alzheimer's disease (AD). A connection exists between serum cholesterol levels and the incidence and the pathophysiology of AD, so the use of compounds that are known to be involved with the lowering of cholesterol may be effective in treating, preventing, and reducing the risk of AD. However, the present inventors have found that the cholesterol-lowering effect alone does not indicate that a compound will have an effect on AP production and/or release. Accordingly, the invention provides methods for selecting agents that have this desired effect on β-amyloid. One such group of compounds are agonists for members of the family of the peroxisome proliferator-activated receptors (PPAR), particularly PPARα and PPARδ.

The peroxisome proliferator-activated receptors (PPARs) [α,δ, β, and γ] are a subfamily of the nuclear receptor gene family (reviewed in Desvergne & Wahli, Endocrine Rev 20:649-688 (1999)). All PPARs are, to various extents, activated by fatty acids and derivatives; PPARα binds the hypolipidemic fibrates whereas antidiabetic glitazones are ligands for PPARδ. PPARα activation mediates pleiotropic effects such as stimulation of lipid oxidation, alteration in lipoprotein metabolism and inhibition of vascular inflammation, to name but a few. PPARα activators increase hepatic uptake and the esterification of free fatty acids by stimulating the fatty acid transport protein and acyl-CoA synthetase expression. In skeletal muscle and heart, PPARα increases mitochondrial free fatty acid uptake and the resulting free fatty acid oxidation through stimulating the muscle-type carnitine palmitoyltransferase-I. The effect of fibrates on the metabolism of triglyceride-rich lipoproteins is due to a PPARα dependent stimulation of lipoprotein lipase and an inhibition of apolipoprotein C-III expression, whereas the increase in plasma HDL cholesterol depends on an overexpression of apolipoprotein A-I and apolipoprotein A-II.

In contrast to PPARα, the function of PPARδ is not well understood. Although PPARδ is ubiquitously expressed the brain, adipose tissue and skin have higher levels of relative mRNA expression (Peters, J. M. et al., Mol. Cell. Biol. 20:5119-5128, 2000). Based on its expression profile, Xing G., et al. (Biochem. Biophys. Res. Commun. 217:1015-1025, 1995) suggest that PPARδ may be involved in brain functions. Furthermore, PPARδ may be implicated in reverse cholesterol transport (Oliver, W. R. et al., Proc. Nat'l. Acad. Sci. 98:5306-5311, 2001). Examples of PPARδ agonists include but are not limited to valproic Acid (Lampen et al., Tox. Appl. Pharmacol. 160:238-249, 1999), GW501516 (Oliver, W. R. et al., Proc. Nat'l. Acad. Sci. 98:5306-5311, 2001), L-165041, L-1 65461, L-783483, and L-796449 (Berger et al., J. Biol. Chem. 274:6718-6725,1999).

For example, the invention provides a method of treatment comprising modulating the production and/or release of β-amyloid in a human in need of said treatment, said method comprising administering to said human a compound of the formula (2) defined as

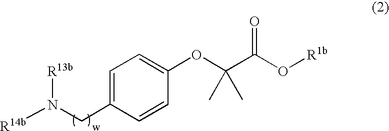

wherein,

R^{1b} is selected from the group consisting of C₁-C₃ alkyl, hydrogen, metal cation and ammonium cation;

R^{13b} and R^{14b} are each independently selected from the group consisting of hydrogen, alkyl, alkenyl, aryl, aralkyl, aralkenyl, cycloalkyl, cycloalkylalkyl, cycloalkylalkenyl, haloalkyl, haloalkenyl, —OR^{12b}, —C(O)OR^{12b}, —N(R^{12b})₂, —C(O)N(R^{12b})₂—N(R^{12b})C(O)OR^{12b}, heterocyclyl and heterocyclylalkyl;

R^{12b} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and

w is 1, 2 or 3.

Specific compounds having PPARα agonist and/or PPARδ agonist activity are compounds having the formula (2) wherein, in one embodiment, R^{1b} is hydrogen, while in another embodiment R^{1b} is a metal cation or an ammonium cation, while in another embodiment R^{1b} is an organic moiety having at least 2, or at least 3, or at least 4, or at least 5, or at least 6 carbons; while in another embodiment R^{1b} enhances the penetration of the compound through the blood brain barrier relative to the corresponding compound wherein R^{1b} is hydrogen, R^{13b} and R^{14b} are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R^{10b}—N═N—O—R^{11b}, —OR^{12b}, C(O)OR^{12b}, —N(R^{12b})₂, —C(O)N(R^{12b})₂, —N(R^{12b})C(O)OR^{11b}, heterocyclyl and heterocyclylalkyl; R^{10b} is a bond dr a straight or branched alkylene or alkenylene chain; R^{11b} is hydrogen, alkyl or aralkyl; R^{12b} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and n is 1, 2 or 3. In various embodiments, R^{1b} is an organic group having less than 30 carbons and a formula weight of less than 1,000, or less than 900, or less than 800, or less than 700, or less than 600, or less than 500. In addition, or alternatively, R^{1b} can be described as being hydrophobic. In addition, or alternatively, R^{1b} is selected from the group consisting of alkyl, alkenyl, aryl, aralkyl, aralkenyl, cycloalkyl, cycloalkylalkyl, cycloalkylalkenyl, halogen, haloalkyl, haloalkenyl, cyano, nitro, —R^{10b}—N═N—O—R^{11b}, —OR^{12b}, —C(O)OR^{12b}, —N(R^{12b})₂, —C(O)N(R^{12b})₂, —N(R^{12b})C(O)OR^{11b}, heterocyclyl and heterocyclylalkyl. In addition, or alternatively, R^{1b} is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4. In addition, or alternatively, R^{1b} is a fragment of insulin wherein said insulin fragment binds to an insulin receptor, for example, said fragment of insulin may consist of: (a) a peptide chain having 14 to 21 amino acid residues from the N-terminus of insulin chain A; and (b) another peptide chain having 16 to 22 amino acid residues from the N-terminus of insulin chain B. In addition, or alternatively, R^{1b} is a protein that binds to a transferrin receptor. In addition, or alternatively, R^{1b} is an antibody or a fragment thereof capable of binding to a ligand in the brain, for example, said antibody may be a monoclonal antibody. In addition, or alternatively, R^{1b} is a growth factor, for example, said growth factor may be EGF.

Other exemplary PPARα agonists consist of the following structure:

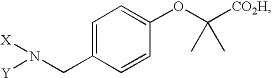

wherein X is selected from the group (a-t) as shown below, and Y is selected from the group (1-8) as shown below.

Exemplary PPARδ agonists consist of the following structure:

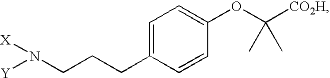

wherein X is selected from the group (a-t) as shown below, and Y is selected from the group (1-8) as shown below.

| X | | | | Y | |
|---|---|---|---|---|---|
| a | 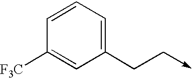 | k | 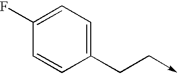 | 1 | 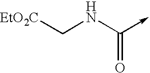 |
| b | 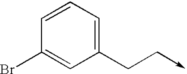 | l | 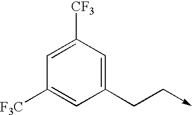 | 2 | 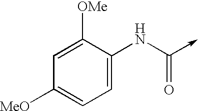 |
| c | 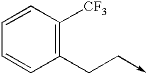 | m | 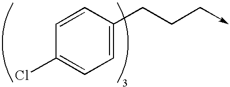 | 3 | 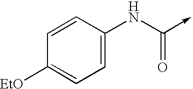 |
| d | 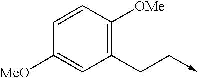 | n | 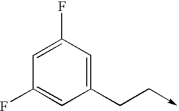 | 4 | 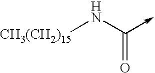 |
| e | 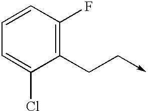 | o | 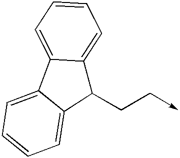 | 5 | 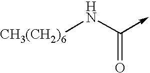 |
| f | 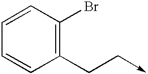 | p | 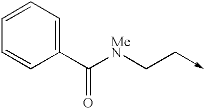 | 6 | 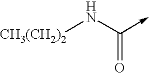 |
| g | 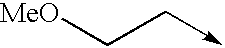 | q | 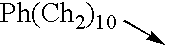 | 7 | 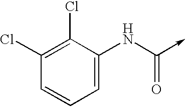 |
| h | 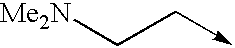 | r | 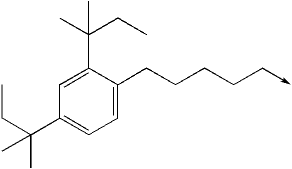 | 8 | 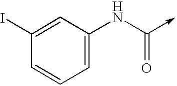 |
| i | 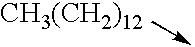 | s | 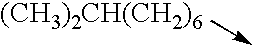 | | |
| j | 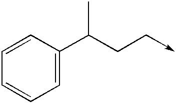 | t | 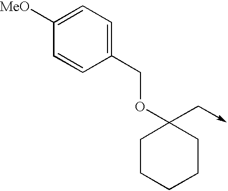 | | |

A preferred member of this group of agonists has the formula

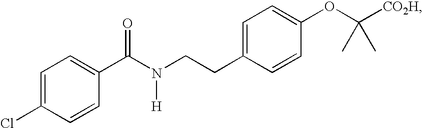

and is also referred to as bezafibrate (Brown, P. J. et al., Chem. And Biol. 4:909-918, 1997), where this compound or esters thereof, i.e., the carboxylic acid of bezafibrate or a reactive equivalent thereof is reacted with an alcohol or a reactive equivalent thereof to form the corresponding ester having an R¹ group, may be used in the methods of the present invention.

Another preferred compound, a PPARδ agonist also disclosed by Brown, P. J. et al., is referred to as 9w2433 and has the following structure:

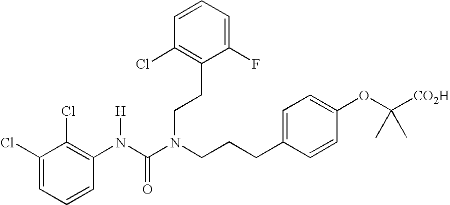

where 9w2433 and esters thereof, i.e., the carboxylic acid of 9w2433 or a reactive equivalent thereof is reacted with an alcohol or a reactive equivalent thereof to form the corresponding ester having an R¹ group, are preferred compounds, and are preferred agents in the methods and compositions disclosed herein.

PPARs are also expressed in atherosclerotic lesions (Bishop-Bailey, Br. J. Pharmacol. 129:823-834, 2000). PPARα is present in endothelial and smooth muscle cells, monocytes and monocyte-derived macrophages. It inhibits inducible nitric oxide synthase in macrophages and prevents the IL-1-induced expression of IL-6 and cyclooxygenase-2, as well as thrombin-induced endothelin-1 expression, as a result of a negative transcriptional regulation of the nuclear factor-kappa B and activator protein-1 signaling pathways. PPAR activation also induces apoptosis in human monocyte-derived macrophages, most likely through inhibition of nuclear factor-kappa B activity. Therefore, the pleiotropic effects of PPARα activators on the plasma lipid profile and vascular wall inflammation likely participate in the inhibition of atherosclerosis development. In addition to lowering cholesterol, according to the present invention, they may also be effective in treating, preventing, and reducing the risk of AD.

The compounds of formula (1) are described, in part, by the presence of various groups, e.g., Y, W, R¹, R², R³, etc., and various integers, e.g., m, n, p, q, etc. The term “independently at each occurrence” in connection with a description of the compound and the various groups and integers thereof is intended to indicate that the selection of the identity for a particular group or integer is independent of the selection of the identity of any other group or integer. Furthermore the selection of any one group at one instance is independent of the selection of the same group at another instance (which will arise when a group, e.g., R¹, appears more than once in the compound). Furthermore, the selection of any one integer (e.g., t) at one occurrence in the compound is entirely independent of the selection of the same integer if and when it occurs an additional time in the compound.

The compounds as set forth above, including any express requirements or express limitations, and any combinations thereof, may be present in a composition of the present invention as described below, and may be used in any of the methods of the present invention as described below. In other words, in describing a method of the present invention that utilizes a compound of formulae (1), (1a), (1b), (1c), (1d) or (2), the compound of the formula may be described in terms of any one or more the express requirements and/or express limitations set forth herein. Likewise with descriptions of compositions of the present invention.

Compound Synthesis

Compounds of formulae (1), (1a), (1b), (1c), or (1d) may be prepared according to methods known to one skilled in the art, or by the methods similar to those disclosed in U.S. Pat. Nos. 3,814,761, 4,559,345 and Gaetano d'Atri, et. al., J. Med. Chem., (1984) 27, 1621-1629, all of which are incorporated in full by reference herein, or by methods similar to the method described below.

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein.

The use of protecting groups is described in detail in Green, T. W. and P. G. M. Wutz, Protective Groups in Organic Synthesis (1999), 3rd Ed., Wiley-Interscience. The protecting group may also be a polymer resin such as a Wang resin or a 2-chlorotrityl chloride resin.

It will also be appreciated by those skilled in the art, although such protected derivatives of compounds of formulae (1), (1a), (1b), (1c), or (1d), as described above in the Summary of the Invention, may not possess pharmacological activity as such, they may be administered to a mammal in need of treatment according to the present invention and thereafter metabolized in the body to form compounds of the invention which are pharmacologically active. Such derivatives may therefore be described as “prodrugs”. All prodrugs of compounds of formulae (1), (1a), (1b), (1c), or (1d) are included within the scope of the invention.

The following Reaction Schemes illustrate methods to make compounds of formula (1b). It is understood that one of ordinary skill in the art would be able to make the compounds of formula (1b) by similar methods or by methods known to one skilled in the art. In general, starting components may be obtained from sources such as Aldrich, or synthesized according to sources known to those of ordinary skill in the art (see, e.g., Smith and March, March's Advanced Organic Chemistry: Reactions, Mechanisms, and Structure, 5th Ed., 2000 (Wiley Interscience, N.Y.)). Moreover, the various R groups (e.g., R¹, R², R³ and R²⁴, etc.) of the compounds of formula (1b) are selected from components as indicated in the description of formula (1b), and may be attached to starting components, intermediate components, and/or final products according to schemes known to those of ordinary skill in the art. R¹, R², R³ and R²⁴ are defined as above. X is Cl or Br. R′ is an alkyl or an aryl group.

Compounds of formula (1b) may be prepared according to the Scheme 1 depicted below.

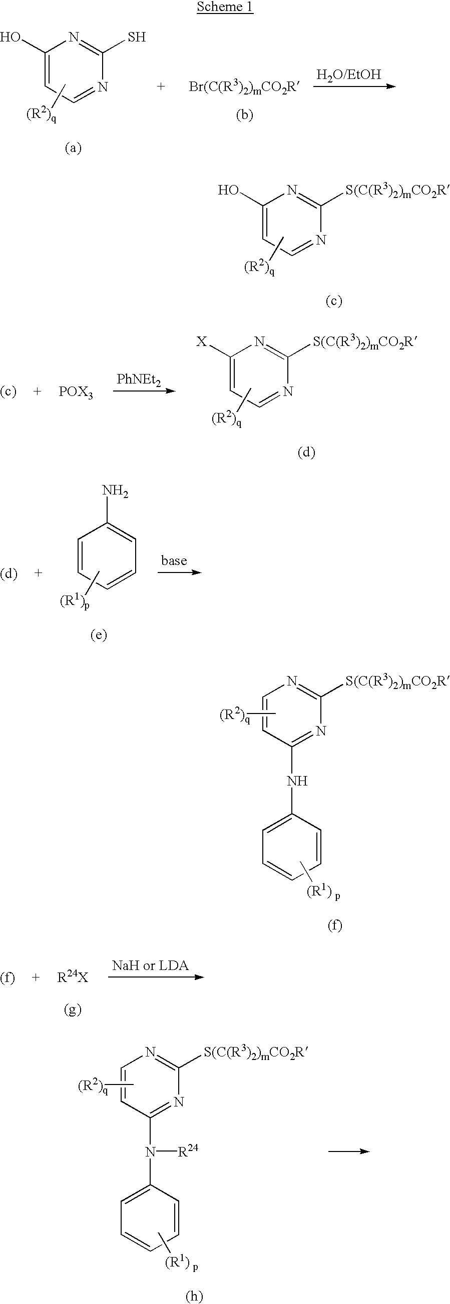

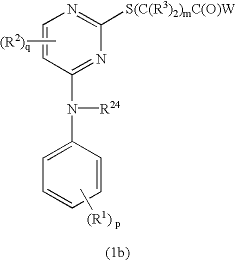

In general, the starting material of formula (a) reacts with a halogenated compound of formula (b) in the presence of a base such as NaHCO₃ at room temperature to afford the compound of formula (c). The conversion of the hydroxyl group to a Cl or Br group is accomplished by treating the compound of formula (c) with an agent such as POCl₃, POBr₃, PCl₅ or PBr₅ and the like under reflux. The compound of formula (d) reacts with primary amine of formula (e) to afford the compound of formula (f). The alkylation of the secondary amine of compound of formula (f) is achieved by reacting the compound of formula (f) with a base such as NaH or LDA or the like at suitable temperature then further reacting with an alkyl halide to afford the compound of formula (h). The transformation of the compound of formula (h) to a compound of formula (1b) can readily be achieved by transesterification, saponification and hydrolysis as well as by amidation of the free carboxyl group or the corresponding acid halide. The conversion of a sulfanyl group of the compound of formula (1b) to a sulfinyl or a sulfonyl group can be achieved by oxidation using a reagent such as H₂O₂ and the like.

Alternatively, compounds of formula (1b) can be prepared as illustrated in Scheme 2.

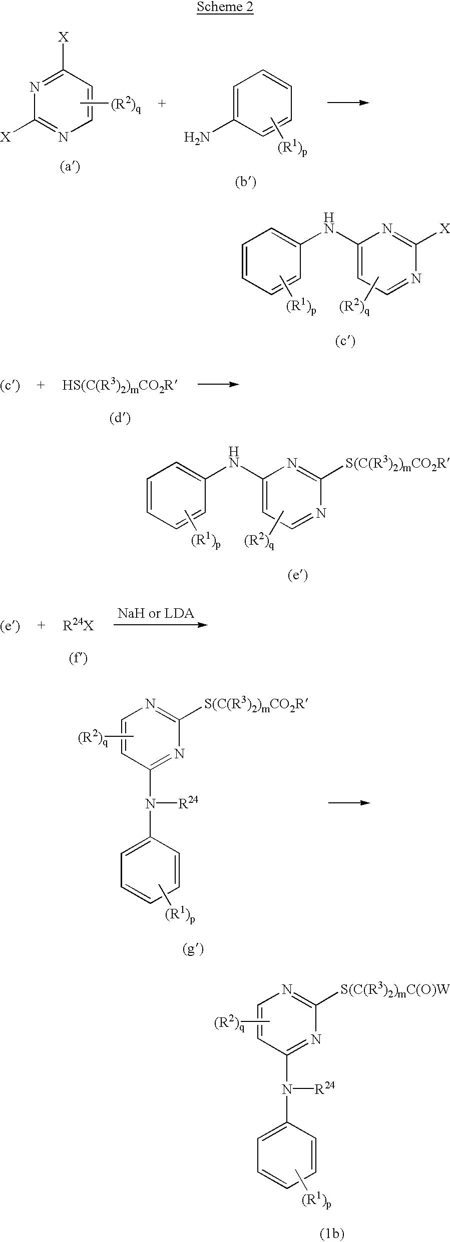

In general, the halogenated starting material of formula (a′) is condensed with a compound of formula (b′) such as phenylamine in an appropriate solvent under reflux to yield the compound of formula (c′) which is then transformed to a compound of formula (e′) by reacting with a compound of formula (d′) in the presence of a base such as Na₂CO₃ and the like under reflux. The product is isolated and dried, and treated with a reagent like NaH or LDA at appropriate temperature and then alkylated by an alkyl halide such as methyl iodide and the like to afford the compound of formula (g′). The transformation of the compound of formula (g′) to a compound of formula (1b) can readily be achieved by transesterification, saponification and hydrolysis as well as by amidation of the free carboxyl group or the corresponding acid halide. The conversion of a sulfanyl group of the compound of formula (1b) to a sulfinyl or a sulfonyl group can be achieved by oxidation using a reagent such as H₂O₂ and the like.

Alternatively, compound of formula (1b) can be prepared as illustrated in Scheme 3. P indicates a protection group such as BOC group and the like. It is more suitable to prepare compounds with R¹ as electron-withdrawing groups, such as —NO₂, —CF₃ and the like.

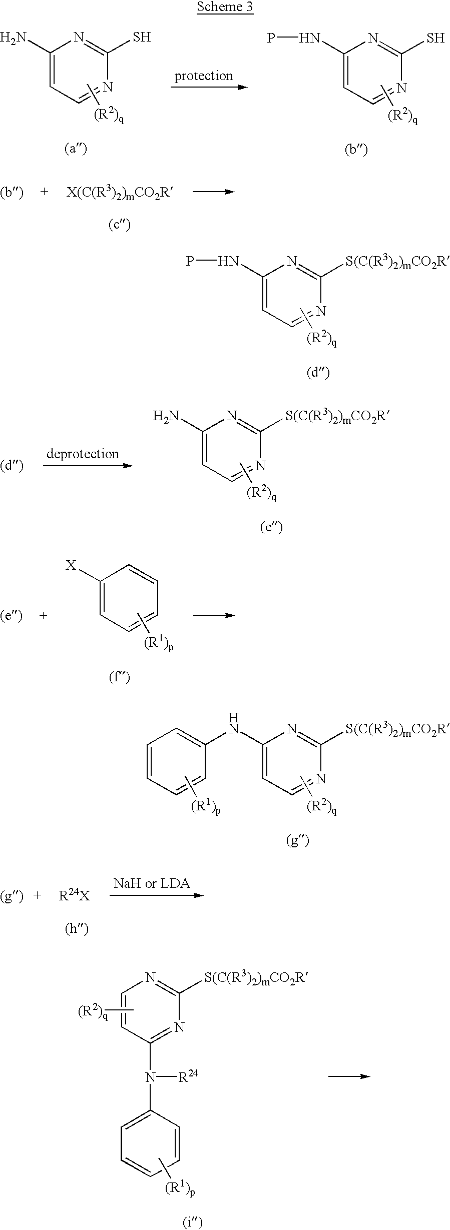

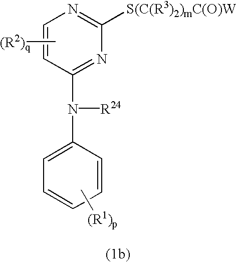

In general, the amino group of a compound of formula (a″) is protected by a protection group such as BOC and the like with the procedure known to those skilled in the art to yield the N-protected product (b″). This N-protected compound is then reacted with a halogenated compound of formula (c″) at room temperature in the presence of a base, such as NaHCO3 and the like to afford the compound of formula (d″). Treatment of the compound of formula (d″) with weak acid such as trifluoroacetic acid to remove the protection group to obtain the amino product of formula (e″). Compound of formula (e″) is then condensed with a compound of formula (f′) such as phenylamine in an appropriate solvent under reflux to yield the compound of formula (g″). The product is isolated and dried, and treated with a reagent like NaH or LDA at appropriate temperature and solvent and then alkylated by an alkyl halide of formula (h″) such as methyl iodide and the like to afford the compound of formula (i″). The transformation of the compound of formula (i″) to a compound of formula (1b) can readily be achieved by transesterification, saponification and hydrolysis as well as by amidation of the free carboxyl group or the corresponding acid halide. The conversion of a sulfanyl group of the compound of formula (1b) to a sulfinyl or a sulfonyl group can be achieved by oxidation using a reagent such as H₂O₂ and the like.

The Aβ-modulating compounds used according to this invention may be readily prepared from (4,6-dichloro-2-pyrimidinylthio) alkanoic acid intermediates which themselves are obtained, for example, by converting 2-thiobarbituric acid to the (4,6-dihydroxy-2-pyrimidinythio)alkanoic acid ester by reaction with an alpha-halo (lower)alkanoic acid ester and subsequently displacing the 4- and 6-positioned hydroxyl groups with chlorine by reaction with an agent such as POCl3, PCl5, and the like. For instance:

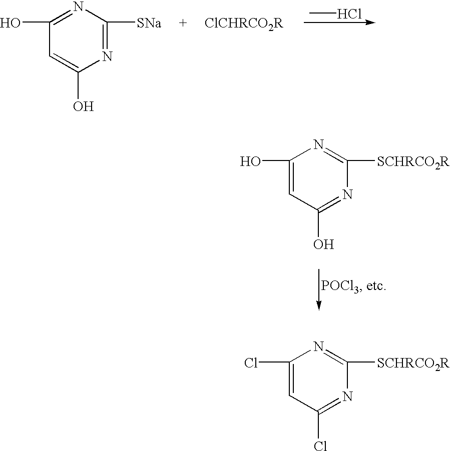

Various modifications of the 4,6-halo groups may be accomplished by substitution and displacement reactions. Thus, reactions of the (4,6-dichloro-2-pyrimidinylthio)alkanoic acid esters with primary amines yields the corresponding 4- or 6-amino derivative, reaction with hydrazine affords the 4- or 6-hydrazino derivative which readily converted to a hydrazone by reaction with an aldehyde or a carbonhydrazide by reaction with a carboxylic acid halide. An aryl group is positioned directly on the 4- or 6-position of the pyrimidine nucleus, if desired, by employing 6-phenyl-2-thiouracil as the initial reactant in lieu of a thio-barbituric acid. From the intermediate monochloro-4 or 6-substituted-2-pyrimidinylthio acetic acid ester, modification of the carboxylic acid functional group is readily achieved by transesterification, saponification and hydrolysis as well as by amidation of the free carboxyl group or the corresponding acid halide.

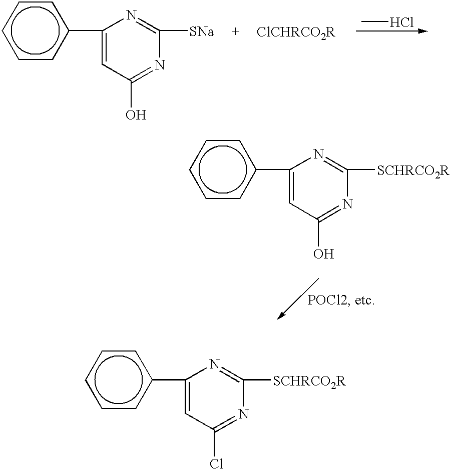

Enhanced Penetration of Blood Brain Barrier

Compounds that may be useful in vitro or in vivo for inhibiting Aβ production and/or release from cells will typically be more effective in alleviating or preventing Aβ production and/or release in the brain if they can gain access to target cells in the brain. A brain cell is defined herein as any cell residing within the skull bone of the head including the spinal cord. Non-limiting examples of brain cells are neurons, glial cells (astrocytes, oligodendrocytes, microglia), cerebrovascular cells (muscle cells, endothelial cells), blood cells (red, white, platelets, etc.) and cells that comprise the meninges. However, access is restricted due to the blood brain barrier (BBB), a physical and functional blockade which separates the brain parenchyma from the systemic circulation (reviewed in Pardridge et al., J Neurovirol 5(6):556-569, 1999; Rubin and Staddon, Rev. Neurosci 22:11-28, 1999). Circulating molecules are normally able to gain access to brain cells via one of two processes: (i) lipid-mediated transport of small molecules through the BBB by free diffusion, or (ii) catalyzed transport. Thus, compounds that are useful for inhibiting Aβ production and/or release are preferably linked to agents that will facilitate penetration of the blood brain barrier. In one embodiment, the method of the present invention will employ a naturally occurring polyamine linked to a small molecule useful at inhibiting Aβ production and/or release. Natural cell metabolites that may be used as linkers, include, but are not limited to, putrescine (PUT), spermidine (SPD), spermine (SPM), or DHA. An alternative method to deliver a compound across the BBB is by intracerebroventricular pump.

The neurologic agent may also be delivered to the nasal cavity. It is preferred that the agent be delivered to the olfactory area in the upper third of the nasal cavity and particularly to the olfactory epithelium in order to promote transport of the agent into the peripheral olfactory neurons rather that the capillaries within the respiratory epithelium. In a preferred embodiment the transport of neurologic agents to the brain is accomplished by means of the nervous system instead of the circulatory system so that small molecules which inhibit Aβ production and/or release may be delivered to the appropriate areas of the brain.

It is preferable that the neurologic agent be capable of at least partially dissolving in the fluids that are secreted by the mucous membrane that surround the cilia of the olfactory receptor cells of the olfactory epithelium in order to be absorbed into the olfactory neurons. Alternatively, the agent may be combined with a carrier and/or other substances that foster dissolution of the agent within nasal releases. Potential adjuvants include GM-1, phosphatidylserine (PS), and emulsifiers such as polysorbate 80.

To further facilitate the transport of the neurologic agent into the olfactory system, the method of the present invention may combine the agent with substances that enhance the absorption of the agent through the olfactory epithelium. It is preferred that the additives promote the absorption of the agent into the peripheral olfactory receptor cells. Because of their role in odor detection, these peripheral neurons provide a direct connection between the brain and the outside environment.

The olfactory receptor cells are bipolar neurons with swellings covered by hair-like cilia which project into the nasal cavity. At the other end, axons from these cells collect into aggregates and enter the cranial cavity at the roof of the nose. It is preferred that the neurologic agent is lipophilic in order to promote absorption into the olfactory neurons and through the olfactory epithelium. Among those neurologic agents that are lipophilic are gangliosides and phosphatidylserine (PS). Alternatively, the neurologic agent may be combined with a carrier and/or other substances that enhance the absorption of the agent into the olfactory neurons. Among the supplementary substances that are preferred are lipophilic substances such as gangliosides and phosphatidylserine (PS). Uptake of non-lipophilic neurologic agents such as nerve growth factor (NGF) may be enhanced by the combination with a lipophilic substance.

In one embodiment of the method of the invention, the neurologic agent may be combined with micelles comprised of lipophilic substances. Such micelles may modify the permeability of the nasal membrane and enhance absorption of the agent. Among the lipophilic micelles that are preferred are gangliosides, particularly GM-1 ganglioside, and phosphatidylserine (PS). The neurologic agent may be combined with one or several types of micelle substances.

Once the agent has crossed the nasal epithelium, the invention further provides for transport of the neurologic agent along the olfactory neural pathway. The agent may be combined with substances that possess neurotrophic or neuritogenic properties which, in turn, may assist in transporting the agent to sites of nerve cell damage. Prophylactic therapies may apply the agent alone or in combination with a carrier, other agents, and/or other substances that may enhance the absorption of the agent into the olfactory neurons.

To deliver the agent to the olfactory neurons, the agent alone or in combination with other substances as a pharmaceutical composition may be administered to the olfactory area located in the upper third of the nasal cavity. The composition may be dispensed intranasally as a powdered or liquid nasal spray, nose drops, a gel or ointment, through a tube or catheter, by syringe, by packtail, by pledget, or by submucosal infusion.

Other modifications of the compounds described herein in order to enhance penetration of the blood brain barrier can be accomplished using methods and derivatives known in the art, including but not limited to those disclosed in the following patent publications, each of which is incorporated by reference herein:

U.S. Pat. No. 6,024,977, issued Feb. 15, 2000 to Yatvin, discloses covalent polar lipid conjugates for targeting to brain and central nervous system.

U.S. Pat. No. 5,017,566, issued May 21, 1991 to Bodor discloses β and γ cyclodextrin derivatives comprising inclusion complexes of lipoidal forms of dihydropyridine redox targeting moieties.

U.S. Pat. No. 5,023,252, issued Jun. 11, 1991 to Hseih discloses the use of pharmaceutical compositions comprising a neurologically active drug and a compound for facilitating transport of the drug across the blood brain barrier including a macrocyclic ester, diester, amide, diamide, amidine, diamidine, thioester, dithioester, thioamide, ketone or lactone.

U.S. Pat. No. 5,024,998, issued Jun. 18, 1991 to Bodor discloses parenteral solutions of aqueous-insoluble drugs with β and γ cyclodextrin derivatives.

U.S. Pat. No. 5,039,794, issued Aug. 13, 1991 to Wier et al. discloses the use of a metastatic tumor-derived egress factor for facilitating the transport of compounds across the blood brain barrier.

U.S. Pat. No. 5,112,863, issued May 12, 1992 to Hashimoto et al. discloses the use of N-acyl amino acid derivatives as antipsychotic drugs for delivery across the blood brain barrier.

U.S. Pat. No. 5,124,146, issued Jun. 23, 1992 to Neuwelt discloses a method for delivery of therapeutic agents across the blood brain barrier at sites of increased permeability associated with brain lesions.

U.S. Pat. No. 5,153,179, issued Oct. 6, 1992 to Eibl discloses acylated glycerol and derivatives for use in a medicament for improved penetration of cell membranes.

U.S. Pat. No. 5,177,064, issued Jan. 5, 1993 to Bodor discloses the use of lipoidal phosphonate derivatives of nucleoside antiviral agents for delivery across the blood brain barrier.

U.S. Pat. No. 5,254,342, issued Oct. 19, 1993 to Shen et al. discloses receptor-mediated transcytosis of the blood brain barrier using the transferrin receptor in combination with pharmaceutical compounds that enhance or accelerate this process.

U.S. Pat. No. 5,258,402, issued Nov. 2, 1993 to Maryanoff discloses treatment of epilepsy with imidate derivatives of anticonvulsive sulfamate.

U.S. Pat. No. 5,270,312, issued Dec. 14, 1993 to Glase et al. discloses substituted piperazines as central nervous system agents.

U.S. Pat. No. 5,284,876, issued Feb. 8, 1994 to Shashoua et al., discloses fatty acid conjugates of dopamine drugs.

U.S. Pat. No. 5,389,623, issued Feb. 14, 1995 to Bodor discloses the use of lipoidal dihydropyridine derivatives of anti-inflammatory steroids or steroid sex hormones for delivery across the blood brain barrier.

U.S. Pat. No. 5,405,834, issued Apr. 11, 1995 to Bundgaard et al. discloses prodrug derivatives of thyrotropin releasing hormone.

U.S. Pat. No. 5,413,996, issued May 9, 1995 to Bodor discloses acyloxyalkyl phosphonate conjugates of neurologically-active drugs for anionic sequestration of such drugs in brain tissue.

U.S. Pat. No. 5,434,137, issued Jul. 18, 1995 to Black discloses methods for the selective opening of abnormal brain tissue capillaries using bradykinin infused into the carotid artery.

U.S. Pat. No. 5,442,043, issued Aug. 15, 1995 to Fukuta et al. discloses a peptide conjugate between a peptide having a biological activity and incapable of crossing the blood brain barrier and a peptide which exhibits no biological activity and is capable of passing the blood brain barrier by receptor-mediated endocytosis.

U.S. Pat. No. 5,466,683, issued Nov. 14, 1995 to Sterling et al. discloses water soluble analogues of an anticonvulsant for the treatment of epilepsy.

U.S. Pat. No. 5,525,727, issued Jun. 11, 1996 to Bodor discloses compositions for differential uptake and retention in brain tissue comprising a conjugate of a narcotic analgesic and agonists and antagonists thereof with a lipoidal form of dihydropyridine that forms a redox salt upon uptake across the blood brain barrier that prevents partitioning back to the systemic circulation.

International Pat. Application Publication Number WO85/02342, published Jun. 6, 1985 for Max-Planck Institute discloses a drug composition comprising a glycerolipid or derivative thereof.

International Patent Application Publication Number WO089/11299, published Nov. 30, 1989 for State of Oregon discloses a chemical conjugate of an antibody with an enzyme which is delivered specifically to a brain lesion site for activating a separately-administered neurologically-active prodrug.

International Patent Application Publication Number WO91/04014, published Apr. 4, 1991 for Synergen, Inc. discloses methods for delivering therapeutic and diagnostic agents across the blood brain barrier by encapsulating the drugs in liposomes targeted to brain tissue using transport-specific receptor ligands or antibodies.

International Patent Application Publication Number WO91/04745, published Apr. 18, 1991 for Athena Neurosciences, Inc. discloses transport across the blood brain barrier using cell adhesion molecules and fragments thereof to increase the permeability of tight junctions in vascular endothelium.

International Patent Application Publication Number WO91/14438, published Oct. 3, 1991 for Columbia University discloses the use of a modified, chimeric monoclonal antibody for facilitating transport of substances across the blood brain barrier.

International Pat. Application Publication Number WO94/01131, published Jan. 20, 1994 for Eukarion, Inc. discloses lipidized proteins, including antibodies.

International Pat. Application Publication Number WO94/03424, published Feb. 17, 1994 for Ishikira et al. discloses the use of amino acid derivatives as drug conjugates for facilitating transport across the blood brain barrier.

International Patent Application Publication Number WO94/06450, published Mar. 31, 1994 for the University of Florida discloses conjugates of neurologically-active drugs with a dihydropyridine-type redox targeting moiety and comprising an amino acid linkage and an aliphatic residue.

International Patent Application Publication Number WO94/02178, published Feb. 3, 1994 for the U.S. Government, Department of Health and Human Services discloses antibody-targeted liposomes for delivery across the blood brain barrier.

International Patent Application Publication Number WO95/07092, published Mar. 16, 1995 for the University of Medicine and Dentistry of New Jersey discloses the use of drug-growth factor conjugates for delivering drugs across the blood brain barrier.

International Patent Application Publication Number WO96/00537, published Jan. 11, 1996 for Southern Research Institute discloses polymeric microspheres as injectable drug-delivery vehicles for delivering bioactive agents to sites within the central nervous system.

International Patent Application Publication Number WO96/04001, published Feb. 15, 1996 for Molecular/Structural Biotechnologies, Inc. discloses omega-3-fatty acid conjugates of neurologically-active drugs for brain tissue delivery.

International Patent Application Publication Number WO96/22303, published Jul. 25, 1996 for the Commonwealth Scientific and Industrial Research Organization discloses fatty acid and glycerolipid conjugates of neurologically-active drugs for brain tissue delivery.

In general, it is well within the ordinary skill in the art to prepare an ester, amide or hydrazide derivative from the corresponding carboxylic acid and a suitable reagent. For instance, a carboxylic acid-containing compound, or a reactive equivalent thereof, may be reacted with a hydroxyl-containing compound, or a reactive equivalent thereof, so as to provide the corresponding ester. The following reference books and treatise provide exemplary reaction conditions to achieve such conversions: “Synthetic Organic Chemistry”, John Wiley & Sons, Inc., New York; S. R. Sandler et al., “Organic Functional Group Preparations,” 2nd Ed., Academic Press, New York, 1983; H, O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 5th Ed., Wiley-Interscience, N.Y., 2000.

One of skill in the art can readily modify any of the compounds discussed above and test them for the desired activity and ability to penetrate the blood brain barrier. For example, the compound of formula (1b) can be modified to improve its blood brain barrier penetration by conjugation to an organic moiety known to cross the blood brain barrier, e.g., docosahexaenoic acid (DHA) and the like. The conjugation of DHA to the compound of formula (1b) can be achieved by following the literature reported procedure. The following references are listed as the examples: Bradley et. al., J. Controlled Release, 74, 233-236, 2001; Katz et al., U.S. Pat. No. 5,716,614; Bradley et al., U.S. Pat. No. 5,955,459; Shashoua et. al., U.S. Pat. No. 5,795,909; Shashoua, U.S. Pat. No. 6,225,444 and U.S. Pat. No. 6,258,836. The conjugation of the compound of formula (1) can be via a hydroxy or an amino group or other function groups which can form a covalent bond with DHA or the like. Scheme 4 is one of the examples for the conjugation of the compound formula (1b) to a DHA molecule via an amino group on the phenyl ring.

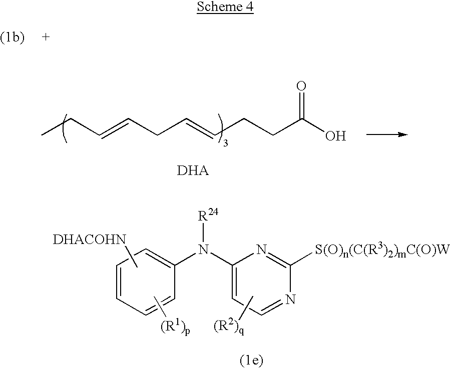

In general, the compound of formula (1e) can be prepared by standard amide formation known to those skilled in the art. The carboxyl group can be converted to an active ester or to an acid chloride or to an anhydride and then the intermediate reacts with the compound of formula (1b) containing an amino group. The DHA conjugated compounds for formulae (1), (1a), (1c) or (1d) containing an amino group can also be prepared similarly.

Transcytosis, including receptor-mediated transport of compositions across the blood brain barrier, is also suitable for the compounds of the invention. Transferrin receptor-mediated delivery is disclosed in U.S. Pat. Nos. 5,672,683; 5,383,988; 5,527,527; 5,977,307; and 6,015,555. Transferrin-mediated transport is also disclosed in Friden, P. M. et al., Pharmacol. Exp. Ther. 278:1491-1498, 1996; and Lee, H.J., J. Pharmacol. Exp. Ther. 292:1048-1052, 2000. EGF receptor-mediated delivery is disclosed in Deguchi, Y. et al., Bioconjug. Chem. 10:32-37, 1999, and transcytosis is described in Cerletti, A. et al., J. Drug Target. 8:435-446, 2000. The use of insulin fragments as carriers for delivery across the blood brain barrier is discussed by Fukuta, M. et al., Pharm. Res. 11:1681-1688, 1994. Delivery of compounds via a conjugate of neutral avidin and cationized human albumin is described by Kang, Y. S. et al., Pharm. Res. 1:1257-1264, 1994.

Although BBB penetration of a therapeutic compound may be desired, recent evidence suggests that BBB penetrable compounds may not necessarily be required to decrease CNS β-amyloid levels. Shibata et al (J Clin Invest 106: 1489-1499, 2000) demonstrate that CSF Aβ can be transported across the BBB into the systemic circulation, thereby decreasing Aβ in the CNS. Once in the systemic circulation, Aβ interacts with binding proteins such as ApoJ/ApoE, which results in a decrease in “free” Aβ in the circulation and shifts the equilibrium to facilitate further transport of Aβ out of the CNS. Thus, the systemic circulation may act as a “sink” or pool of Aβ that can regulate CNS β-amyloid levels (Shibata, M et al., J Clin Invest 106: 1489-1499, 2000). This “peripheral sink” hypothesis is supported by vaccination studies with anti-Aβ antibodies in AD transgenic mouse models. For example, vaccination of PDAPP mice with an Aβ antibody (m266) resulted in accumulation of CNS derived Aβ in the plasma (DeMattos et al., PNAS 98: 8850-8855, 1998; Holtzman et al., Adv Drug Delivery Rev 54: 1603-1613, 2002). Therefore, if compounds can systemically decrease Aβ levels, the peripheral sink hypothesis indicates that this may shift the Aβ equilibrium between the CNS and plasma resulting in a decreased β-amyloid burden in the CNS. Therefore, pharmaceutical agents of the invention can act systemically and may not be required to cross the BBB.

Nevertheless, in one aspect of the invention, a compound of formula (1) is conjugated to another compound in order to provide an agent, where the agent has enhanced ability to cross the BBB relative to the compound of formula (1). Methods of conjugating a biologically active agent to a compound, and suitable compounds that upon conjugation to a biologically active agent provide a conjugate having enhanced ability to cross the BBB, are well known from the above-cited references, and these same techniques may be applied to effectively enhance the permeability of compounds of formula (1) to the BBB.

Pharmaceutical Compositions and Administration

The compounds of this invention can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

In pharmaceutical dosage forms, the compounds may be administered in the form of their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoro-methane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at ambient temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well known in the art. Implants are formulated as microspheres, slabs, etc. with biodegradable or non-biodegradable polymers. For example, polymers of lactic acid and/or glycolic acid form an erodible polymer that is well tolerated by the host. The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Depending on the patient and condition being treated and on the administration route, the specific compounds may be administered in dosages of 0.1 μg to 40 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus for example oral dosages in the rat may be ten times the injection dose. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

For use in the subject methods, the subject compounds may be formulated with other pharmaceutically active agents known to one of ordinary skill in the art. The compounds may be administered to an individual suffering from Alzheimer's disease in unit doses containing from about 0.01 to 1000 milligrams of active ingredient, the remainder of the formulation constituting known adjuvants. The goal of the therapy is modulation of β-amyloid production and/or release. This modulation can be by one or more chemically induced physiological mechanisms.

In human treatment, from 1 to 40 milligram, or 1 to 10 milligram, and conventionally 5 milligram doses of the active compounds of this invention are considered to be most desirable from the standpoint of uniform presentation for controlled administration. The compounds of the invention may be administered alone or in combination with pharmacologically acceptable carriers, the proportion of which is determined by the chosen route of administration and standard pharmaceutical practice. For example, they may be administered orally in tablet or capsule form with conventional flavors, diluents, lubricants, disintegrators or binding agents as may be required. They may be administered orally in the form of a solution or they may be injected parenterally. For parenteral administration they may be used in the form of a sterile solution containing other solutes, for example, enough saline or glucose to make the solution isotonic.

A suitable formulation for parenteral administration is as follows:

| | |
|---|---|
| Sodium[4-chloro-6-(2,3-xylidino)-2- | 5 mg |
| pyrimidinylthio] acetate | |
| Vehicle: sterile water, containing benzyl alcohol | 5 ml |
| (1 percent) and sodium acetate-acetic acid | |
| buffer 0.6% | |

Methods of Use

The compounds described above may be tested for their effect on Aβ release using in vitro tests. Routine experimentation can also be performed to determine if a composition affects the release of Aβ from at least one cell in vivo. Other suitable assays are disclosed in the Examples herein. Briefly, SM-4 cells, which are stably transfected with Swedish mutant β-amyloid Precursor Protein, are treated with a PPARα and/or PPARδ agonist, such as pirinixic acid, or derivative thereof. After treatment, the media is collected and assayed for Aβ40 and/or Aβ42. A statistically significant decrease (p<0.05) in Aβ40 or Aβ42 concentration in the media compared to appropriate control(s) indicates that the treatment inhibited or prevented Aβ40 and/or Aβ42 production and/or release from the cells. If a compound decreases Aβ42 production and/or release by a statistically significant amount relative to control (absence of the compound or presence of vehicle) it is considered to be an Aβ42-modulating agent according to the invention.

There is a complex relationship between AD, cholesterol homeostasis, and agents used for regulating cholesterol levels in the body. WO 00/28981 discloses the administration of an inhibitor of HMG CoA reductase (3-hydroxy-3-methylglutaryl CoA reductase) to reduce the risk of onset of Alzheimer's disease. The inhibitors used were lovastatin, pravastatin, or a combination thereof. However, a similar correlation was not seen with simvastatin. WO 00/31548 also discloses inhibitors of HMG CoA reductase, particularly statins. Interestingly, simvastatin is a suggested inhibitor, contrasting with the results disclosed in WO 00/28981, which states that the prevalence of AD in simvastatin-treated patients was not decreased.

Fassbender, K. et al., PNAS/www.pnas.org/cgi/doi/10.1073/-pnas.081620098, describe the use of simvastatin to reduce levels of β-amyloid peptides Aβ42 and Aβ40 in vitro and in vivo, using guinea pigs. Wolozin, B. et al., Arch. Neurol. 57:1439-1443, 2000, describe the analysis of a patient population treated with HMG-CoA reductase inhibitors. The authors reported that the prevalence of AD was 60-73% lower in these patients than in patients taking other medications. In this study, a causal relationship could not be established. Jick, H. et al., The Lancet 356:1627-1631, 2000, also reviewed patient records and found that in individuals 50 years and older, statin administration was associated with a substantially lowered risk of dementia, including Alzheimer's disease and other conditions. Similarly, Acyl-CoA: cholesterol acyltransferase (ACAT) inhibitors have been used to decrease plasma cholesterol in various animal models including rats, guinea pigs and rabbits (Tanaka et al., J. Med. Chem. 41:2390-2410, 1998; Junquero et al., Biochem. Pharmacol. 61:97-108, 2001). Examples of ACAT inhibitors include but are not limited to Glibenclamide, CI-976 (PD128042), NTE-122, Fatty acid Anilides, F12511, Avasimibe, TS-962 (HL-004), N-Chlorosulfonyl isocyanate and derivatives, SR-9223i, Pyripyropenes, PD-132301, PD-132301-2, DUP-128, YM-17E, BW447A, AD 6591, CL-277,082, Melinamide, Hydroxyphenyl Urea derivatives, R-106578, Indoline derivatives with amide or urea moiety, 57-118, 58-035, CI-999, CI-1011, N-alkyl-N-[(fluorophenoxy)benzyl]-N′-arylureas and derivatives, SKF-99085, EAB309, N-alkyl-N-(heteraryl-substituted benzyl)-N′-arylureas and derivatives, F-1394, N-alkyl-N-biphenyllylmethyl-N′-aryl ureas and derivatives, CL 277,082, CL 283,546, CL 283,796, CP-113,818, CP-105,191, Polyacetylene analogs-panaxynol, panaxydol, panaxydiol and panaxytriol, T-2591, 4,4-bis(trifluoromethyl)imidazolines and derivatives, FR145237, FR186054, FR129169, Naringenin, Ulmoidol, 23-hydroxyursolic acid, 27-trans-p-coumaroyloxyursolic acid, 27-cis-p-coumaroyloxyursolic acid, Triterpenes and derivatives, N-(4,5-diphenylthiazol-2-yl)-N′-aryl or alkyl (thio)ureas and derivatives, N-(4,5-diphenylthiazol-2-yl)alkanamides and derivatives, RP73163, RP64477, Diaryl-substituted heterocyclic ureas and derivatives, Heterocyclic amides and derivatives, Cyclic sulfides derived from hetero-Diels-Alder reaction of thioaldehydes with 1,3-dienes, E5324, Tetrazole amide derivatives of (+/−)-2-dodecyl-alpha-phenyl-N-(2,4,6-trimethoxyphenyl)-2H-tetrazole-5-acetamide, Epi-cochlioquinone A, Acyclic(diphenylethyl) diphenylacetamides, 2-(1,3-Dioxan-2-yl)-4,5-diphenyl-1H-imidazoles and derivatives, N-(2,2-dimethyl-2,3-dihydrobenzofuran-7-yl)amide derivatives, FCE 27677, GER1-BP002-A, TMP-153, Amides of 1,2-diarylethylamines and derivatives, F-1394, N-(4-oxochroman-8-yl)amide derivatives, Terpendoles, Short chain ceramide and dihydroceramide, FY-087, 447C88, Cyclandelate, 3-quinolylurea derivatives, N-phenyl-6,11-dihydrodibenz[b,e]oxepin-11-carboxamides and related derivatives, Gypsetin, AS-183, AS-186, 2,6-disubstituted-3-imidazolylbenzopyrane derivatives, Lateritin, 2-(Alkylthio)-4,5-diphenyl-1H-imidazoles derivatives, Glisoprenins, Acaterin, U-73482, Purpactins, and Chlorpromazine.

An exemplary compound according to the invention is known as pirinixic acid. According to the examples herein, pirinixic acid induced a decrease in Aβ42 production and/or release from SM-4 cells in a concentration-dependent manner. Although pirinixic acid is well known, the present invention is the first disclosure of its use to reduce AP production and/or release. Pirinixic acid has been identified as a hypolipidemic agent, and was first disclosed in U.S. Pat. No. 3,814,761 (Jun. 4, 1974), which characterized it and related compounds as anti-lipidemic agents. Although it might be tempting to view the activity of pirinixic acid on Aβ42 production and/or release as being directly related to its hypolipidemic role, particularly in view of the clinical correlation between hypercholesterolemia and Alzheimer's disease (reviewed in Wolozin, Proc Natl Acad Sci 98:5371-5373 (2001)), in fact the mechanisms appear to be separate. Thus, a cholesterol-lowering agent is not by definition a suitable treatment for AD without further experimentation, as discussed more fully below.

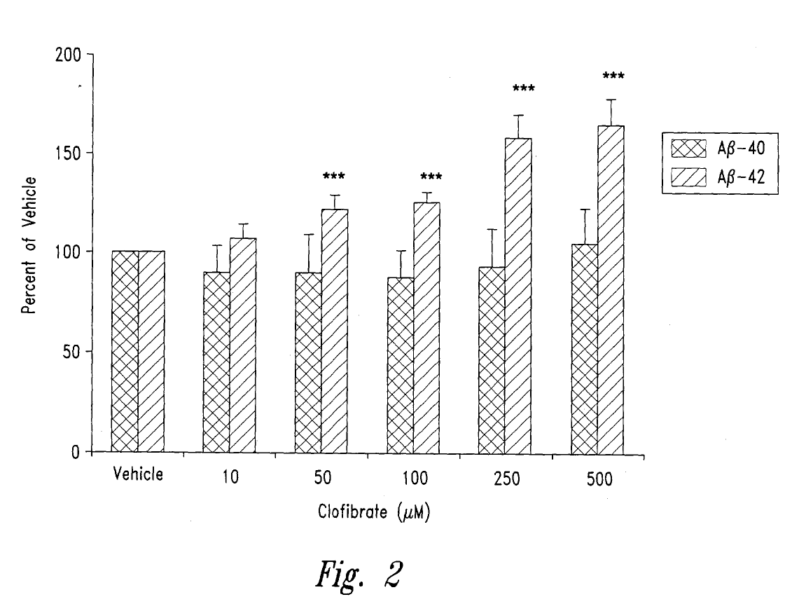

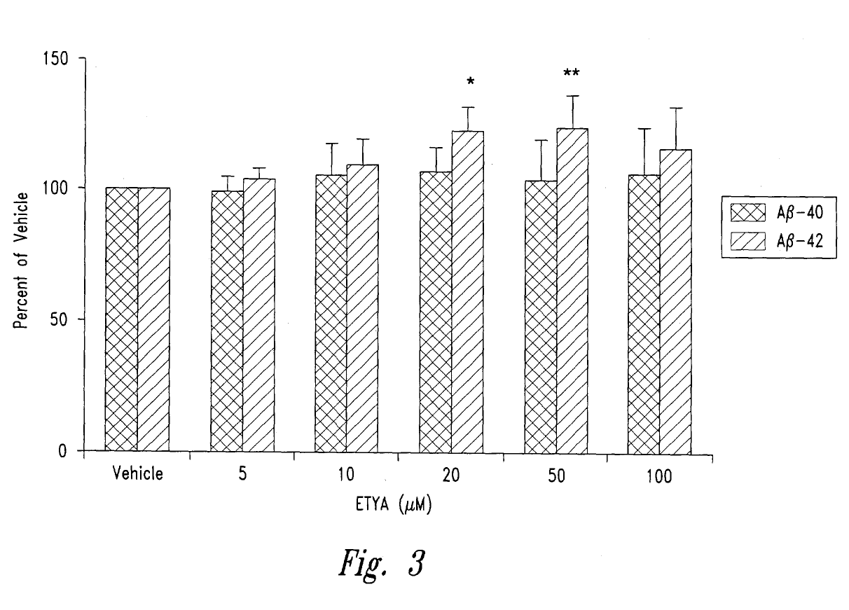

Fibrates are often used as cholesterol-lowering agents but do not generally reduce Aβ42 production and/or release. For example, SM-4 cells were treated with clofibrate and the culture media was collected in order to assay Aβ42 levels. As shown in FIG. 2, clofibrate significantly increased Aβ42 extracellular levels at a concentration range of 50-500 μM. Similar results were found with ETYA at 20-50 μM concentrations, as shown in FIG. 3. The fact that three PPARα agonists (all of which are cholesterol lowering agents) have disparate effects on Aβ42 production and/or release from SM-4 cells supports the premise of the invention, which is that some PPARα agonists affect Aβ42 production and/or release via a mechanism that is not strictly concomitant with their role as cholesterol lowering agents.

The invention therefore relates to the agents pirinixic acid and other PPARα and/or PPARδ agonists, which are capable of reducing Aβ42 production and/or release, wherein the agent is constituted as a pharmaceutical composition, and the agent may or may not be coupled to a carrier, for example as discussed above for promoting penetration of the blood brain barrier.

The compounds and pharmaceutical compositions of the invention are administered to a subject having a pathology associated with increased accumulation or deposition of the β-amyloid peptide such as but not limited to Alzheimer's disease. The present compounds are useful for prophylactic or therapeutic purposes. The prevention of Aβ accumulation and deposition is accomplished by administration of a compound of formula (1) prior to development of overt disease, e.g., to prevent β-amyloid production, release and/or accumulation in the form of plaques, etc. Alternatively the compounds are used to treat ongoing disease, by stabilizing or improving the clinical symptoms of the patient.

The term “subject” is intended to include mammals having β-amyloid production and/or release, including one or more β-amyloid related symptoms, or which are susceptible to β-amyloid production and/or release. Exemplary subjects include, for example, primate sp., particularly humans; rodents, including mice, rats and hamsters; guinea pigs; rabbits; equines, bovines, canines, felines; etc. Animal models are of interest for experimental investigations, providing a model for treatment of human disease. The subject may also be referred to as the host, or the patient, may be from any mammalian species.

One method to identify a subject in need of treatment according to the present invention is to measure cognitive, behavioural and/or memory abilities of the subject. If a subject displays impairment in cognitive functioning, particularly if the subject's cognitive ability declines over time, then the subject may benefit from treatment according to the present invention. If the subject is a human, then cognitive function and impairment indicative of probable Alzheimer's disease can be assessed using psychological and other tests known to those skilled in the art. If the human subject displays characteristics consistent with a disease caused by increased accumulation and/or deposition of the β-amyloid peptide, such as but not limited to Alzheimer's disease, then the subject may benefit from the treatment according to the present invention.

The susceptibility of a particular cell to treatment with the subject compounds may be determined by in vitro testing. Typically a culture of the cell is combined with a compound of formula (1) at varying concentrations for a period of time sufficient to allow the active agents to decrease production and/or release of Aβ, usually between about one hour and one week. For in vitro testing, cultured cells from a biopsy sample may be used.

The dose will vary depending on the specific compound utilized, specific disorder, patient status, etc. Typically a therapeutic dose will be sufficient to produce a substantial decrease β-amyloid production and/or release in the targeted tissue, while maintaining patient viability. Treatment will generally be continued until there is a substantial reduction, e.g., at least 5%, or in another embodiment at least 10%, in β-amyloid levels and may be continued chronically.

The invention provides PPARα and/or PPARδ agonists and derivatives thereof for use in lowering β-amyloid levels, and thereby alleviating, treating, and/or preventing disease associated with buildup of P-amyloid, such as Alzheimer's disease. According to the invention, an exemplary PPARα agonist, pirinixic acid, is useful in reducing Aβ42 production and/or release from cells. By inhibiting Aβ42 production and/or release, buildup of Aβ42 and formation of plaques may be reduced or prevented. These results are consistent with current models for the role of Aβ in Alzheimer's disease. However, not all PPARa agonists can be used for lowering β-amyloid production and/or release. For example, the PPARα agonists ETYA and Clofibrate were found to increase the production and/or release of the Aβ42 from cells, as shown in FIGS. 2 and 3 and as discussed in detail in the examples. These results demonstrate that the definition of a compound as a PPARα agonist is not the only factor that determines an efficacious response (i.e., a decrease in Aβ production and/or release). Rather, the response appears to be specific to the chemical structure. A novel aspect of the invention is the provision of methods and materials for screening PPARα and/or PPARδ agonists and related compounds and derivatives to determine their suitability for modulating Aβ production and/or release from cells in vivo.

Thus, in one aspect, the present invention provides a method for modulating the production and/or release of β-amyloid in a cell, comprising treating said cell with a compound of formula (1), or formula (1a), or formula (1b), or formula (1c), or formula (1d), or formula (2). In optional embodiments: the cell is a brain cell; and/or the β-amyloid is β-amyloid 42; and/or β-amyloid production and/or release in the cell is reduced; and/or the cell is treated in vitro.

In another aspect, the present invention provides a method of treatment comprising modulating the production and/or release of β-amyloid in a non-human mammal in need of said treatment, said method comprising administering to said non-human mammal an effective amount of a compound of formula (1), or formula (1a), or formula (1b), or formula (1c), or formula (1d), or formula (2). In optional embodiments: the cell is a brain cell; and/or β-amyloid is β-amyloid 42; and/or the non-human mammal is a mouse, rat, cat, dog or guinea pig; and/or β-amyloid production and/or release is reduced.

In another aspect, the present invention provides a method of treatment wherein the production and/or release of β-amyloid is modulated in a human in need of said treatment, said method comprising administering to said human an effective amount of a compound of formula (1), or formula (1a), or formula (1b), or formula (1c), or formula (1d), or formula (2). In optional embodiments: the human is afflicted with Alzheimer's disease; and/or the human has suffered a head injury; and/or the human has a genetic predisposition or environment exposure that increases the likelihood that said person will develop Alzheimer's disease; and/or the human exhibits minimal cognitive impairment suggestive of early stage Alzheimer's disease; and/or the production and/or release of the β-amyloid is a brain cell is modulated; and/or the β-amyloid is β-amyloid 42; and/or β-amyloid production and/or release is reduced.

As mentioned above, the method of the present invention may preferentially reduce production and/or release of Aβ42 relative to one or more other forms of Aβ, in a target that produces and/or releases Aβ42, for instance a target selected from a cell, a human, a non-human mammal, and the brain of a human. Tests to identify the selectively of such a compound are disclosed herein. Thus, in one aspect, the present invention provides that a subject in need to selective reduction of Aβ42 relative to one or more other forms of Aβ, is administered a compound of formula (1), or formula (1a), or formula (1b), or formula (1c), or formula (1d), or formula (2) that affords such selectively.

The invention is further directed to a pharmaceutical composition comprising an amount of a compound as disclosed herein, or a neurologic agent, which is effective in treating or preventing brain disorders such as Alzheimer's disease, when administered thereto, in combination with a pharmaceutically acceptable vehicle such as a liquid or powdered carrier and/or various optional adjuvants.

In one embodiment, the invention provides method of treatment comprising modulating the production and/or release of β-amyloid in a non-human mammal in need of said treatment. In another embodiment, the invention provides method of treatment comprising modulating the production and/or release of β-amyloid in a human in need of said treatment. Whether the treating is to human or non-human mammals, the inventive method comprises administering to said subject a compound or composition as described herein, and particularly a compound selected from compounds of the formulae (1), (1a), (1b), (1c), (1d) and (2), each as defined herein, including various embodiments thereof.

The invention is also described with reference to the following examples, which are not intended to be limiting. All patents and publications referenced above and in the Examples are incorporated by reference herein.

### EXAMPLES

Preparation 1

### Synthesis of [4-chloro-6-(2,3-dimethylphenylamino)pyrimidin-2-ylsulfanyl]acetic Acid Ethyl Ester

To a solution of NaHCO₃ (8.4 g, 0.1 mole) in 500 mL of water was added 2-thiobarbituric acid (14.4 g, 0.1 mol) with stirring. Ethyl bromoacetate (11.1 mL, 0.1 mol) was then added and followed by the addition of 400 mL of EtOH to obtain a clear solution.

This mixture was kept stirring at room temperature for 3 hours and then the solvent was removed in vacuum and a precipitate was formed. The solid was collected by filtration, washed with the mother liquor, and then dried in vacuum over P₂O₅ for 3 days to yield 17.9 g (78%) of the white solid product which was used in the next step reaction without further purification.

To a mixture of the white solid obtained above (17.9 g, 77.7 mmol) in 120 mL of POCl₃ was slowly added N,N-diethylaniline (11.6 g, 77.7 mmol) at 5° C. over 10 minutes. The mixture was stirred at 10-15° C. for 15 minutes and then refluxed for 5 hours. The excess POCl₃ was removed in vacuum. The residue was treated with cold water (500 mL) and the mixture was stirred for 3 days and then filtered. The solid collected was recrystallized from hexanes yielding 6.73 g (32%) of the product which was used for the next step reaction without further purification.

A mixture of the solid obtained above (1.33 g, 5 mmol), 2,3-dimethylaniline (0.63 mL, 5.16 mmol) and Na₂CO₃ (0.55 g, 5.24 mmol) in 25 mL of EtOH was refluxed for 19.5 hours. The solvent was removed in vacuum and the residue was purified by flash column chromatography (silica gel,1^{st} eluted with EtOAc:hexanes=1:1 and 2^{nd} eluted with Et₂O:hexanes=1:4). The desired product was obtained in 26% yield (0.456 g) and used for next step reaction without further purification.

### Example 1

### Synthesis of {4-chloro-6-[(2,3-dimethylphenyl)ethylamino]pyrimidin-2-yl-sulfanyl}acetic Acid (Compound 2)

To a solution of NaHCO₃ (8.4 g, 0.1 mole) in 500 mL of water was added 2-thiobarbituric acid (14.4 g, 0.1 mol) with stirring. Ethyl bromoacetate (11.1 mL, 0.1 mol) was then added and followed by the addition of 400 mL of EtOH to obtain a clear solution. This mixture was kept stirring at room temperature for 3 hours and then the solvent was removed in vacuum and a precipitate was formed. The solid was collected by filtration, washed with the mother liquor, and then dried in vacuum over P₂O₅ for 3 days to yield 17.9 g (78%) of the white solid product which was used in the next step reaction without further purification.

To a mixture of the white solid obtained above (17.9 g, 77.7 mmol) in 120 mL of POCl₃ was slowly added N,N-diethylaniline (11.6 g, 77.7 mmol) at 5° C. over 10 minutes. The mixture was stirred at 10-15° C. for 15 minutes and then refluxed for 5 hours. The excess POCl₃ was removed in vacuum. The residue was treated with cold water (500 mL) and the mixture was stirred for 3 days and then filtered. The solid collected was recrystallized from hexanes yielding 6.73 g (32%) of the product which was used for the next step reaction without further purification.

A mixture of the solid obtained above (1.33 g, 5 mmol), 2,3-dimethylaniline (0.63 mL, 5.16 mmol) and Na₂CO₃ (0.55 g, 5.24 mmol) in 25 mL of EtOH was refluxed for 19.5 hours. The solvent was removed in vacuum and the residue was purified by flash column chromatography (silica gel,1 st eluted with EtOAc:hexanes=1:1 and 2^{nd} eluted with Et₂O:hexanes=1:4). The desired product was obtained in 26% yield (0.456 g) and used for next step reaction without further purification.

To a solution of [4-chloro-6-(2,3-dimethylphenylamino)pyrimidin-2-ylsulfanyl]-acetic acid ethyl ester (0.63 g, 1.79 mmol) in 6 mL of THF was added NaH (60%, 90 mg, 2.24 mmol) at 15° C. The mixture was kept stirring at 15° C. for about 40 minutes and then iodoethane (0.37 g, 2.38 mmol) was added. Stirring was continued at room temperature for 22 hours. The reaction was quenched by the addition of silica gel (in Et₂O:hexanes=1:6) and EtOAc (ethyl acetate, 10 mL). The solvents were removed and the residue was purified by flash column chromatography eluted with Et₂O:hexanes=1:6. The alkylated product was obtained in 67% yield (0.456 g).

To a hot solution of the product obtained above (0.452 g, 1.19 mmol) in 6 mL of EtOH was added NaOH solution (1 M, 3 mL). The mixture was heated in an oil bath (˜95° C.) for 6.5 minutes, and then diluted with 25 mL of water. EtOH was removed by evaporation in vacuo. The aqueous layer was acidified with conc. HCl to pH ca. 1-2 and then extracted with Et₂O (3×25 mL). The combined organic layers were dried over anhydrous Na₂SO₄, filtered and evaporated to yield 0.418 g of crude product which was purified by flash column chromatography (silica gel, MeOH:CH₂Cl₂=1:9) to afford 0.411 g of the pure product as a white solid (98% yield). ¹H NMR (ppm, CDCl₃): 1.20 (t, 3H, J=7.0 Hz), 2.04 (s, 3H), 2.30 (s, 3H), 3.51-3.63 (m,1H), 3.86 (s, 2H), 3.93-4.27 (m,1H), 5.58 (s, 1H), 6.92-6.94 (m, 1H), 7.20-7.28 (m, 2H). ¹³C NMR (ppm, CDCl₃): 12.5, 14.0, 20.5, 33.9, 45.1, 99.5, 126.2, 127.3, 130.3, 134.6, 139.5, 139.6, 158.0, 162.1, 170.2, 172.4. MS (m/z, ES+): 352.0 (100%, M+1).

### Example 2

### Synthesis of {4-chloro-6-[(2,3-dimethylphenyl)methylamino]pyrimidin-2-ylsulfanyl}acetic acid (compound 1)

This compound as a white solid was obtained in a manner analogous that that described in Example 1. ¹H NMR (ppm, CDCl₃): 2.04 (s, 3H), 2.35 (s, 3H), 3.41 (s, 3H), 3.84 (s, 2H), 5.68 (s,1H), 6.95-6.98 (m,1H), 7.20-7.30 (m, 3H). MS (m/z, ES+): 338.0 (100%, M+1).

### Example 3

### Synthesis of {4-[(2,3-dimethylphenyl)propylamino]-6-chloropyrimidin-2-ylsulfanyl}acetic Acid

To a cold solution (−78° C.) of [4-chloro-6-(2,3-dimethylphenylamino)-pyrimidin-2-ylsulfanyl]acetic acid ethyl ester (0.144 g, 0.41 mmol) in 1.5 mL of anhydrous THF was slowly added lithium diisopropylamide (LDA, 2.0 M, 0.21 mL) over 20 minutes. This mixture was kept stirring at −78° C. for 15 minutes before 0.3 mL of HMPA was added. Twenty minutes after the addition of HMPA, 1-iodopropane was added and the mixture was kept stirring at −78° C. for 2 hours before it was slowly warmed to room temperature (over about 1.5 hours). The reaction was quenched by the addition of 10 mL of water after being cooled to 0° C. The mixture was extracted with EtOAc (3×10 mL). The combined organic layers were dried over Na₂SO₄, the volatile solvent(s) were removed in vacuo and the residue was purified by flash column chromatography eluted with Et₂O:hexanes=1 :2. The product was obtained in 89% yield (0.144 g) and used without further purification.

To a hot solution of the product obtained above (0.140 g, 0.355 mmol) in 2 mL of EtOH was added NaOH solution (1 M, 1 mL). The mixture was heated in an oil bath (˜95° C.) for 6.5 minutes, and then diluted with 10 mL of water. EtOH was removed by evaporation in vacuo. The aqueous layer was extracted with Et₂O (2×10 mL) and the organic layers were discarded. The aqueous layer was acidified with conc. HCl to pH ca. 1-2 and then extracted with Et₂O (3×15 mL). The combined organic layers were dried over anhydrous Na₂SO₄, filtered and evaporated. The residue was purified by flash column chromatography (silica gel, MeOH:CH₂Cl₂=1 :9) to afford 0.112 g of the pure product as a white solid (87% yield). ¹H NMR (ppm, CDCl₃): 0.94 (t, 3H, J=7.2 Hz), 1.61-1.71 (m, 2H), 2.05 (s,1H), 2.37 (s, 3H), 3.39-3.47 (m,1H), 3.81 (s, 2H), 4.11-4.19 (m,1H), 5.61 (s,1H), 6.93-6.96 (m,1H), 7.21-7.26 (m, 2H). MS (m/z, ES+): 366.0 (100%, M+1).

### Example 4

### Synthesis of {4-[(2,3-dimethylphenyl)butylamino]-6-chloropyrimidin-2-ylsulfanyl}acetic Acid

This compound as a white solid was obtained in a manner analogous to that described in Example 9. ¹H NMR (ppm, CDCl₃): 0.93 (t, 3H, J=7.2 Hz), 1.34-1.38 (m, 2H), 1.58-1.1.63 (m, 2H), 2.04 (s,1H), 2.37 (s, 3H), 3.43-3.51 (m,1H), 3.85 (s, 2H), 4.15-4.23 (m,1H), 5.58 (s,1H), 6.93-6.96 (m,1H), 7.20-7.26 (m, 2H). MS (m/z, ES+): 380.0 (100%, M+1).

### Example 5

### Synthesis of [4-(2,3-dimethylphenylamino)pyrimidin-2-ylsulfanyl]acetic Acid

The title compound was prepared similarly as described in Example 7 starting from 2-mercaptopyrimidin-4-ol. ¹H NMR (ppm, DMSO-d₆): 2.06 (s, 3H), 2.27 (s, 3H), 3.82 (s, 2H), 6.18 (m, 1H), 7.05-7.14 (m, 3H), 7.99 (m, 1H), 9.12 (s, 1H), 12.60 (s, 1H). MS (m/z, ES+): 290.0 (100%, M+1).

### Example 6

### Effect of Pirinixic Acid Treatment on β-amyloid production and/or Release from Cells

Cell Lines and Pharmacological Treatments. 293 EBNA cells (InVitrogen, Carlsbad, Calif.) stably transfected with Swedish mutant β-Amyloid Precursor Protein -695 (SM4 cells) were routinely maintained in DMEM supplemented with sodium pyruvate (1 mM) and 10% fetal bovine serum. Cells were seeded into poly-D-Lysine (SIGMA) coated 6-well plates at a density of 5-7×10⁵ cells per well. Subsequently, the cells were rinsed in 1 ml of PBS and treated with 10-500 μM of pirinixic acid in serum-free/phenol red-free DMEM for 16 hours.

Aβ Detection and Standardization. After the pharmacological treatment, the exposure media was collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ40 or Aβ42 by a calorimetric ELISA as per the manufacturer's protocol (Biosource International Inc, California). The cells were lysed in 0.1% Triton X-100 in PBS supplemented with 5 μM propridium iodide (Molecular Probes, Eugene, Oreg.) and incubated at 37° C. for 30 minutes prior to measuring fluorescence. Aβ40 and Aβ42 were standardized against propridium iodide fluorescence as a measure of total cell number.

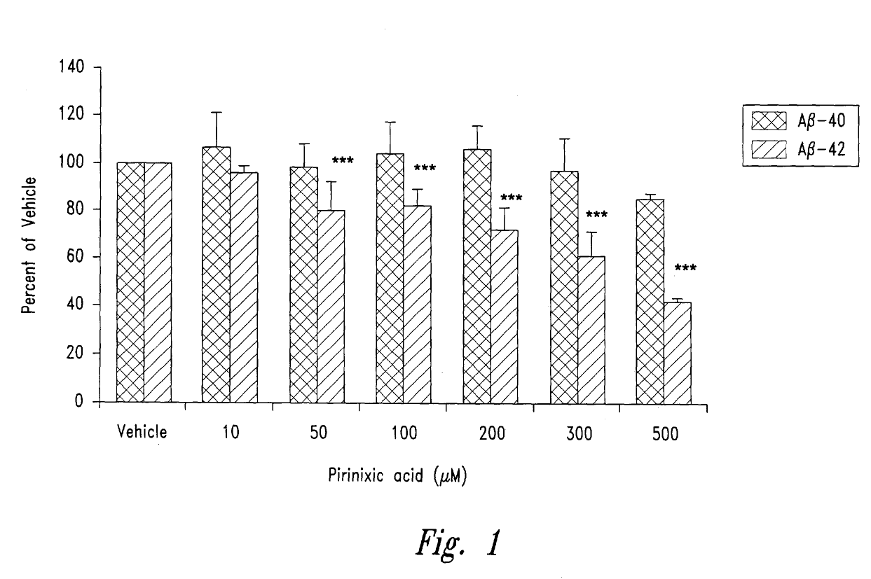

The PPARα and/or PPARδ agonist, pirinixic acid induced a significant decrease in Aβ42 production and/or release from SM-4 cells after 16 hrs. Concentrations as low as 50 μM induced a 15% decrease (p<0.001) in Aβ42. At 500 μM a 60% decrease in Aβ42 was observed (FIG. 1). Interestingly, the pirinixic acid mediated decrease in Aβ production and/or release was selective since there was no significant change in Aβ40 production and/or release.

### Example 7

### Effect of Pirinixic Acid Treatment on Production of Amyloid Precursor Protein and Proteolytic Fragments Thereof

Cell Lines and Pharmacological Treatments. SM4 cells were routinely maintained, seeded into Poly-D-Lysine (SIGMA) coated 6-well plates, rinsed in PBS, and treated with 50-500 μM of pirinixic acid in serum free/phenol red free DMEM for 16 hours as described in Example 6.

Detection of Amyloid Precursor Protein and its Proteolytic Fragments.

After the pharmacological treatment, the conditioned media was harvested and the cellular lysate was collected in 100 μl of cold SAPK lysis buffer (0.01% Nonidet P-40, 20 mM MOPS 5 mM EDTA and 75 mM β-glycerol phosphate, protease inhibitor cocktail (Boehringer Mannheim, Laval, QC)) and sonicated on ice for 8 seconds using a probe sonicator. From each sample, total protein concentration was determined using the bicinchonic acid assay (Pierce, Rockford, II, USA). Cellular APP and secreted APP_{Sα} levels were quantitated by 10% Tris-Glyine SDS-PAGE Western blot analysis using an anti-APP N-terminal antibody (22C11, Boehringer Mannheim, Laval, QC) (Mills et al., 1997; Connop et al., 1999) and monoclonal 6E10 (Senetek Research, Maryland Heights, Mo., USA), respectively. C99 was quantitated from the cellular lysate by 16.5% Tris-Tricine SDS-PAGE Western blot analysis using monoclonal antibody 6E10 (Senetek Research, Maryland Heights, Mo., USA). Immunoreactive bands were visualized using ECL detection (Amersham, Oakville, ON) and analyzed by standard densitometric techniques.

Statistical Analysis. Statistical significance was determined using an ANOVA with Tukey's post hoc analysis. Data are expressed as mean±SD with * p<0.05 and **p<0.01 and n=4.

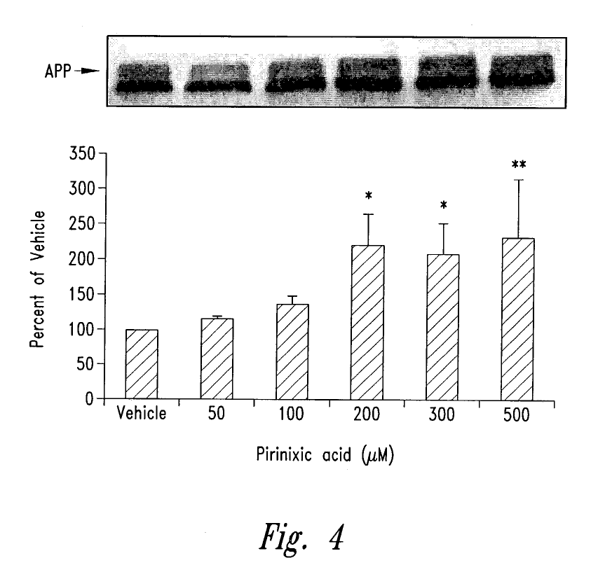

Result. FIG. 4 shows the effect of PPARα and/or PPARδ agonist pirinixic acid on cellular APP levels from SM-4 cells quantitated by Western blot analysis. A representative micrograph of the C99 Western blot data is depicted above the corresponding densitometric values. Data are expressed as mean±SD with n=4 and statistical significance determined by ANOVA with Tukey's post hoc test at *p<0.05 and **p<0.01.

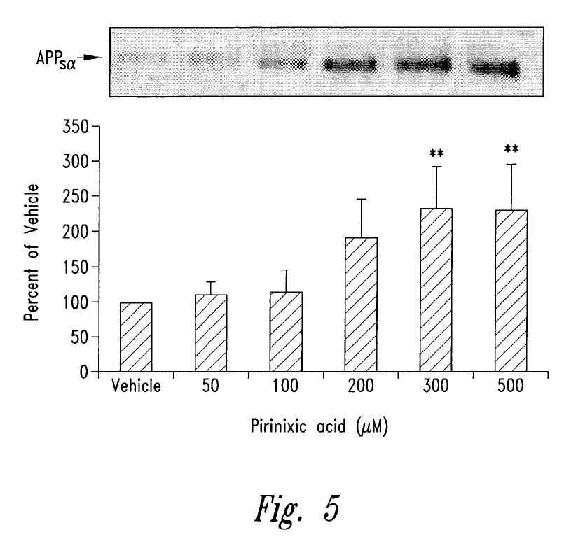

FIG. 5 shows the effect of PPARα and/or PPARδ agonist pirinixic acid on APP_{Sα} release from SM-4 cells quantitated by Western blot analysis. A representative micrograph of the C99 Western blot data is depicted above the corresponding densitometric values. Data are expressed as mean±SD with n=4 and statistical significance determined by ANOVA with Tukey's post hoc test at **p<0.01.

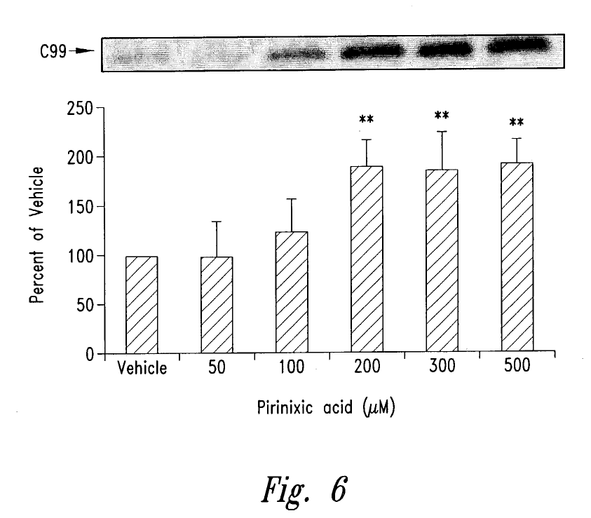

FIG. 6 shows the effect of PPARα and/or PPARδ agonist pirinixic acid on C99 levels from SM-4 cells quantitated by Western blot analysis. A representative micrograph of the C99 Western blot data is depicted above the corresponding densitometric values. Data are expressed as mean±SD with n=4 and statistical significance determined by ANOVA with Tukey's post hoc test at **p<0.01.

### Example 8

### Effect of Compound 1 Treatment on β-Amyloid Production and/or Release from Cells

Cell Lines and Pharmacological Treatments.

293 EBNA cells (InVitrogen, Carlsbad, Calif.) stably transfected with Swedish mutant β-Amyloid Precursor Protein -695 (SM4 cells) were routinely maintained in DMEM supplemented with sodium pyruvate (1 mM) and 10% fetal bovine serum. Cells were seeded into poly-D-Lysine (SIGMA) coated 6-well plates at a density of 5-7×10⁵ cells per well. Subsequently, the cells were rinsed in 1 ml of PBS and treated with 50-300 μM Compound 1 for 16 hrs in serum-free/phenol red-free DMEM.

Aβ Detection and Standardization.

After the pharmacological treatment, the exposure media was collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ40 or Aβ42 by a calorimetric ELISA as per the manufacturer's protocol (Biosource International Inc, California). The cells were lysed in 0.1% Triton X-100 in PBS supplemented with 5 μM propridium iodide (Molecular Probes, Eugene, Oreg.) and incubated at 37° C. for 30 minutes prior to measuring fluorescence. Aβ40 and Aβ42 were standardized against propridium iodide fluorescence as a measure of total cell number.

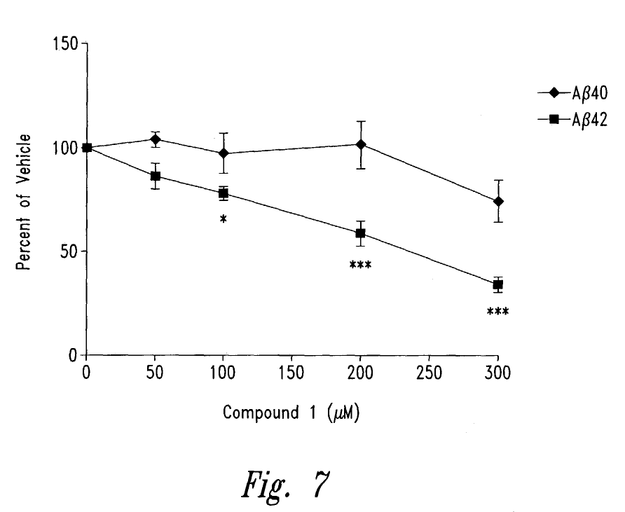

As seen in the FIG. 7, Compound 1 selectively decreased Aβ42 from SM-4 cells in vitro without altering Aβ40. A 66% inhibition of Aβ42 secretion was seen at a concentration of 300 μM Compound 1 (p<0.001).

### Example 9

### Effect of Compound 2 Treatment on β-Amyloid Production and/or Release from Cells

Cell Lines and Pharmacological Treatments.

293 EBNA cells (InVitrogen, Carlsbad, Calif.) stably transfected with Swedish mutant β-Amyloid Precursor Protein -695 (SM4 cells) were routinely maintained in DMEM supplemented with sodium pyruvate (1 mM) and 10% fetal bovine serum. Cells were seeded into poly-D-Lysine (SIGMA) coated 6-well plates at a density of 5-7×10⁵ cells per well. Subsequently, the cells were rinsed in 1 ml of PBS and treated with 5-100 μM Compound 2 for 16 hrs in serum-free/phenol red-free DMEM.

Aβ Detection and Standardization.

After the pharmacological treatment, the exposure media was collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ40 or Aβ42 by a colorimetric ELISA as per the manufacturer's protocol (Biosource International Inc, California). The cells were lysed in 0.1% Triton X-100 in PBS supplemented with 5 μM propridium iodide (Molecular Probes, Eugene, Oreg.) and incubated at 37° C. for 30 minutes prior to measuring fluorescence. Aβ40 and Aβ42 were standardized against propridium iodide fluorescence as a measure of total cell number.

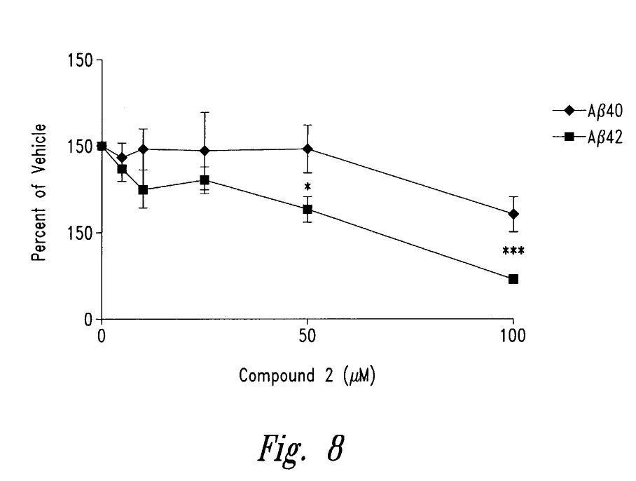

As seen in FIG. 8, Compound 2 selectively decreased Aβ42 without altering Aβ40. An 80% inhibition of AP42 secretion was seen at a concentration of 100 μM Compound 2 (p<0.001).

### Example 10

### Screening Agents for Ability to Decrease β-Amyloid Production and/or Release from Cells

Cell Lines and Pharmacological Treatments.

293 EBNA cells stably transfected with Swedish mutant β-Amyloid Precursor Protein -695 are maintained in DMEM supplemented with sodium pyruvate (1 mM) and 10% fetal bovine serum. Cells are seeded into Poly-D-Lysine coated 6-well plates at a density of 5-7×10⁵ cells per well. Subsequently, the cells are rinsed in 1 ml of PBS and treated with 10-500 μM of a PPARα or a PPARδ agonist in serum-free/phenol red-free DMEM for 16 hours.

Aβ Detection and Standardization.

After the pharmacological treatment, the exposure media is collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ40 or Aβ42 by a calorimetric ELISA as per the manufacturer's protocol (Biosource International, Inc., California). The cells are lysed in 0.1% Triton X-100 in PBS supplemented with 5 μM propridium iodide (Molecular Probes, Eugene, Oreg.) and incubated at 37° C. for 30 minutes prior to measuring fluorescence. Secreted Aβ40 and Aβ42 are standardized against propridium iodide fluorescence as a measure of total cell number.

### Example 11

### Screening Agents for Ability to Decrease β-Amyloid Production Using an In Vitro Gamma Secretase Assay

Several assays have been described in the literature which measure the formation of various Aβ species using an in vitro γ-secretase assay (Tian et al., 8^{th} Intl Conference on Alzheimer's Disease and Related Disorders, Abstract 653, Stockholm, Sweden, 2002; Golde et al., 32^{nd} Annual Society for Neuroscience Conference, Abstract 722.6, Orlando, USA, 2002 Eriksen et al., 32^{nd} Annual Society for Neuroscience Conference, Abstract 722.7, Orlando, USA, 2002). These in vitro assays measure proteolytic activity due to the activity of the y-secretase complex and are known to those skilled in the art. Compounds of formula (1) may be screened using such assays in order to identify their relative ablility to modulate β-amyloid formation.

### Example 12

### The Effect of Pirinixic Acid on Aβ40/42 Production and/or Secretion from Human Neuroblastoma Cells

Cell Lines and Pharmacological Treatment

Human neuroblastoma cells (hDAT; SK-N-MC stably overexpression human dopamine transporter) were routinely maintained in DMEM supplemented with sodium pyruvate (1 mM) and 10% fetal bovine serum. Cells were seeded into 6-well plates at a density of 2.5×10⁵ cells per well and transiently transfected with APPsw (Swedish mutant β-amyloid precursor protein-695) using lipofectamine (Life Technologies, Rockville, Md.) as per the manufacturer's suggested protocol. Subsequently, 48 hours post-transfection the cells were rinsed with PBS and treated with vehicle (0.1% DMSO) or 100-200 μM pirinixic acid in serum free/phenol free DMEM for 24 hours.

Aβ Detection and Standardization

After the pharmacological treatment, the exposure media was collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ40 or AP42 by a calorimetric ELISA as per the manufacturer's protocol (Biosource International Inc, California). The cells were lysed in 0.1% Triton X-100 in PBS supplemented with 5 μM Propridium Iodide (Molecular probes, Eugene, Oreg.) and incubated at 37° C. for 30 minutes prior to measuring fluorescence. Secreted Aβ40 and Aβ42 levels were standardized against propridium iodide fluorescence as a measure of total cell number.

Statistical Analysis

Data are expressed as a percent of control and represent the mean±SD with n=11 and statistical significance determined by ANOVA with a Tukey's post hoc test at ***p<0.001.

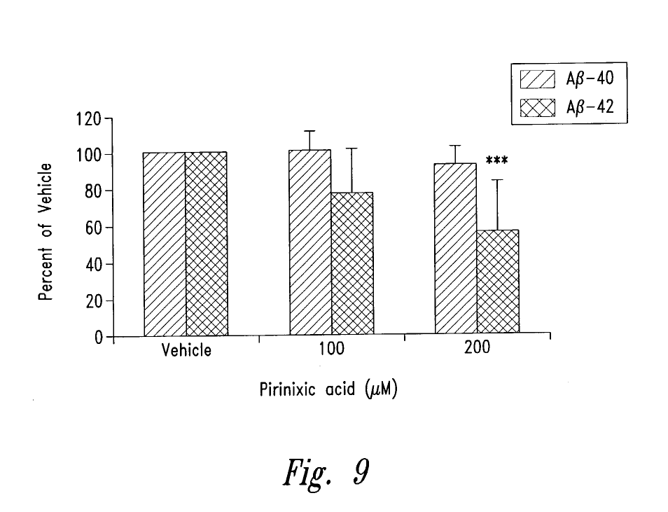

FIG. 9 demonstrates the effects of PPARα and/or PPARδ agonist pirinixic acid on Aβ40/42 from human neuroblastoma cells transiently transfected with APPsw. A concentration of 200 μM pirinixic acid selectively decreases Aβ42 by 40% (p<0.001, n=11) without altering Aβ40.

### Example 13

### The Effect of Pirinixic Acid on AβTotal and Aβ42 Production and/or Secretion from Primary Murine Cortical Neurons

Semliki Forest Virus (SFV) Stocks

The cDNA coding for human APP695 is cloned in the Smal site of pSFV-1 as described previously (Simons et al., J. Neurosci. 16:899-908, 1996; Tienari et al., Embo. J. 15:5218-29, 1996). PSFV-1/huAPP695 constructs are linearized,with SpeI and run-off transcription using SP6 polymerase is performed to produce mRNA. The transcribed mix of APP and pSFV-helper are cotransfected into BHK cells by electroporation to yield recombinant SFV (Olkkonen et al., J. Neurosci. Res. 35:445-51, 1993). BHK cells are grown in DMEM/F12 supplemented with 5% fetal calf serum, 2 mM L-glutamine, 100 U/ml penicillin, and 100 mg/ml streptomycin. Twenty-four hours after transfection, the culture supernatant containing infective recombinant SFV is collected. Aliquots are snap-frozen in liquid nitrogen and stored at −70° C. until use.

Neuronal Culture

All experiments are conducted on murine primary cortical neurons derived from E14 embryos according to established procedures (Annaert et al., J. Cell Biol. 147:277-294, 1999; Cupers et al., J. Cell Biol. 154:731-40, 2001; De Strooper et al., Nature 391:387-90, 1998). Briefly, cortices of 14-day-old murine embryos are dissected, transferred to Hanks' Balanced Salt Solution (HBSS, Gibco BRL, Rockville, Md.) and trypsinized for 15 minutes at 37° C. Dissociated cell suspensions are routinely plated on poly-L-lysine (1 mg/ml, Sigma, St. Louis, Mo.) coated dishes (Nunc, Naperville, Ill.) in Minimal Essential Medium (MEM; Gibco BRL) supplemented with 10% horse serum and transferred to a CO₂ incubator. After 3 hours, the culture medium is replaced by serum-free neurobasal medium with B27 supplement (Gibco BRL). After 24 hours, cytosine arabinoside (5 μM) was added to each dish to prevent normeuronal (glial) cell proliferation. Three to four days post-plating, mixed cortical neuron cultures are used for drug testing.

Semliki Forest Virus Infection

Cortical neurons are incubated with increasing concentrations of pirinixic acid (stock solution 400 mM in DMSO). First, a concentrated dilution series is prepared in DMSO comprising 4, 20, 40 and 200 mM compound. From each of these solutions, 2.5 μl is added to the neuronal cultures in 2 ml of neurobasal medium (dilution 1/800) resulting in 5, 25, 50 and 250 μM final concentrations. As a control, 2.5 μl of DMSO is added to one dish.

After various incubation times at 37° C., the medium is replaced by 1.2 ml neurobasal medium and cultures are transduced by adding recombinant pSFV-humAPP695 wt (dilution 1/10) for 1 hour to allow viral entry. Following a 2-hour incubation in the absence of virus, cultures are metabolically labeled using methionine-free neurobasal medium containing 100 μCi [³⁵S]-methionine (ICN). After 4 hours at 37° C., the conditioned medium and the cell extracts are collected and centrifuged (14,000 rpm, 15 min).

Detection of AβTotal from Conditioned Media

The cleared fractions are subject to immunoprecipitation with antibodies on protein G-Sepharose (Pharmacia). Aβtotal is examined from the cleared conditioned media by immunoprecipitation using pab B7, directed against the first 17 amino acids of Aβ (De Strooper et al., Embo. J. 14:4932-8,1995). After overnight rotation, the immunoprecipitates are washed 5 times in extraction buffer and once in TBS. The bound material is denatured in sample buffer and subject to gel electrophoresis on precast 4-12% Nupage gels. Densitometric analysis is conducted using a Phosphoimager (Molecular Dynamics) and ImagQuant 5.0. Aβtotal levels are normalized to APP levels to control for plate-to-plate variation.

Quantification of Aβ42 by ELISA

The levels of the longer Aβ42 peptide are quantified in both the conditioned media and cell extracts using a sandwich ELISA test (De Strooper et al., Nature 391:387-90, 1998; Vanderstichele et al., Amyloid. 7:245-58, 2000). In summary, 800 μl of conditioned medium or cell extract is lyophilized (Savant Speedvac concentrator), dried pellets are dissolved in 400 μl of sample diluent and applied on a 96-well ELISA plate precoated with the capturing anti-Aβ42 mab 21 F12. This antibody only recognizes the final two amino acids of the Aβ42 sequence. After washing, the wells are incubated with biotin-labeled mAb 3D6 directed against the first 7 amino acids of Aβ, followed by streptavidin-HRP. Finally HRP substrate is added and the colorimetric reaction is quantitated spectrophotometrically using a Victor 2 (Wallac) equipped with a 450 nm filter. For each experiment a duplicate standard curve for Aβ42 is included. The Aβ42 concentrations in the samples are finally calculated based on the Aβ42 standards nonlinear regression equation and using Mathematica 4.1 software package (Wolfram Research, Champaign, Ill.).

Statistical Analysis

Data are expressed as a percent of control and represent the mean±SD with n=6 and statistical significance determined by ANOVA with a Tukey's post hoc test at **p<0.01, ***p<0.001.

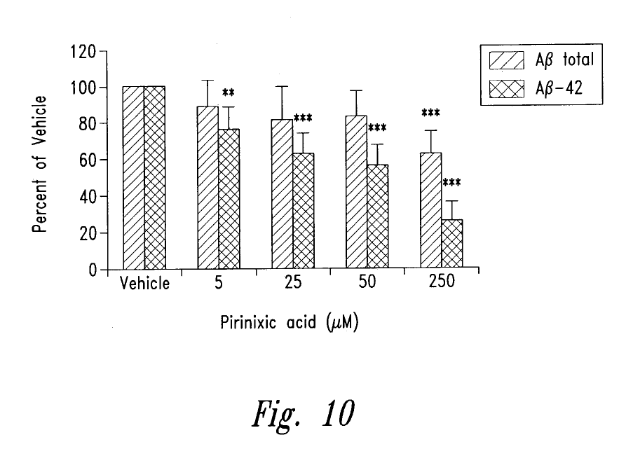

FIG. 10 demonstrates the effects of PPARα and/or PPARδ agonist pirinixic acid on Aβtotal and Aβ42 levels from primary murine cortical neurons infected with APP695. A concentration dependant decrease in Aβ42 was observed. A 20% decrease in Aβ42 was observed at 5 μM pirinixic acid (p<0.01, n=6). In contrast, no significant effect on Aβtotal was observed until cells were treated with 250 μM pirinixic acid. This data demonstrates a selective decrease in Aβ42 at 5-50 μM pirinixic acid without altering Aβtotal.

### Example 14

### Screening Agents for Ability to Decrease β-Amyloid Production and/or Release In Vivo

Upon arrival of the animals from the vendor, adult guinea pigs are housed under alternating 12 hr light/dark cycles with free access to water and food (standard laboratory chow diet). After 5-6 days adjustment to the new environment guinea pigs are anaesthetized with sodium pentobarbital and using standard sterotaxic surgical procedures, the left lateral cerebral ventricle is cannulated. After the minor surgery, the guinea pigs are given an analgesic (Bupivaicane), allowed to recover and monitored to ensure normal behavior (i.e., regular food and water intake, regular rest/activity cycles etc.). One day post-surgery, 25 μl of various doses of compounds of formula (1) diluted in phenol free DMEM supplemented with 6% DMSO are injected into the cannula. Control animals are injected with 25 μl of phenol-free DMEM supplemented with 6% DMSO. Subsequently, at various time points post-injection, CSF is extracted through standard cisterna magna puncture and supplemented with 10% sample treatment buffer (40 mM sodium phosphate (pH 7.4), 40 mM triethanolamine, 0.1% Triton X-100, 200 mM NaCl, 2 mM EGTA, 0.1% sodium azide), prior to freezing. After the protocol has been completed, the guinea pigs are euthanized using lethal injection of sodium pentobarbital. CSF Aβ40 and Aβ42 levels are analyzed by a colorimetric ELISA as per the manufacturers protocol (Biosource International Inc, California).

The guinea pig animal model is only one of several models known in the art that could be used. Other examples include but are not limited to, AD transgenic mice models expressing various forms of APP (Tg2576; TgAPP/Sw/1 TgAPP/Ld/2, PDAPP), presenilins or combinations of both (Tg2576 plus mutant PS1, Tg Hu/MoAPP plus PS1) (Games, D. et al., Nature 373:523-527, 1995; Hsiao, K. H. et al., Science 274: 99-102, 1996; Moechars, D. et al., J. Biol. Chem. 274: 6483-6492, 1999; Holcomb, L. et al., Nature Medicine 4: 97-100, 1998; Borchelt, D. R. et al., Neuron 19: 939-945, 1997).

### Example 15

### Screening Agents for Ability to Penetrate Blood Brain Barrier

Using an in vitro model such as that disclosed in Franke, H. et al., Brain Res. Prot. 5:248-256, 2000, or an in vivo model such as those described by Shulkin, B. L. et al., J. Neurochem. 64:1252-1257, 1995; Thorne, R. G. et al., Brain Res. 692:278-282, 1995; Pan, W., et al., Neuropharmacol. 37:1553-1561, 1998, pharmaceutical agents of the invention can be routinely tested for their ability to penetrate the blood brain barrier. The in vitro model uses a PBEC (porcine brain microvessel endothelial cell) monolayer which is arranged so that the ability of substances to pass from a donor compartment to an acceptor compartment can be measured. This model reflects the in vivo situation wherein substances reach the brain compartment from a brain microvessel. Permeation properties of an agent of the invention are measured by radiolabeling the agent, for example with ³H, and adding it to the donor compartment. Samples are collected from the donor and acceptor compartments at routine intervals and permeability is calculated as described in Franke, H. et al., (2000).

The in vivo models measure the brain influx index or the measure of the passage of a substance through the blood brain barrier. The agent is radiolabeled or fluorescently labeled and administered peripherally by intravenous injection (Pan, W., et al., Neuropharmacol. 37:1553-1561, 1998), orally (Shulkin, B. L. et al., J. Neurochem. 64:1252-1257, 1995) or nasally (Thorne, R. G. et al., Brain Res. 692:278-282, 1995) and the concentration of the agent in the blood as compared to the brain is monitored.

### Example 16

### Screening Agents Administered Systemically that Decrease CNS β-Amyloid Levels

Recent evidence indicates that BBB penetrable compounds may not be required to decrease CNS β-amyloid levels. Shibata et al (J Clin Invest 106: 1489-1499, 2000) demonstrate that CSF Aβ can be transported across the BBB into the systemic circulation, thereby decreasing Aβ in the CNS. Once in the systemic circulation, Aβ interacts with binding proteins such as ApoJ/ApoE, which results in a decrease in “free” Aβ in the circulation and shifts the equilibrium to facilitate further transport of Aβ out of the CNS. Thus, the systemic circulation may act as a “sink” or pool of AP that can regulate CNS β-amyloid levels (Shibata, M et al., J. Clin Invest 106: 1489-1499, 2000). This “peripheral sink” hypothesis is supported by vaccination studies with anti-AP antibodies in AD transgenic mouse models. For example, vaccination of PDAPP mice with an Aβ antibody (m266) resulted in accumulation of CNS derived Aβ in the plasma (DeMattos et al., PNAS 98: 8850-8855, 2001; Holtzman et al., Adv Drug Delivery Rev 54: 1603-1613, 2002). Therefore, if compounds can systemically decrease Aβ levels, the peripheral sink hypothesis indicates that this may shift the Aβ equilibrium between the CNS and plasma resulting in a decreased β-amyloid burden in the CNS. Therefore, pharmaceutical agents of the invention can act systemically and may not be required to cross the BBB.

Using transgenic animal models described above pharmaceutical agents of the invention can be examined for their effects on systemic and CNS β-amyloid levels. Compounds can be injected into the animal of interest followed by repeated sampling and measurement of plasma β-amyloid levels over time. An increase in plasma Aβ levels coupled with a decrease in CNS levels would indicate that the compound is shifting the Aβ equilibrium. Furthermore, the ability of the compound to cross the blood brain barrier in vivo can be measured by standard analytical chemistry techniques (e.g., mass spectroscopy).

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for modulating the production and/or release of β-amyloid in a cell, comprising treating said cell with a compound of formula (1). 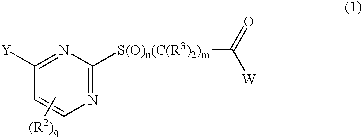
wherein, independently at each occurrence,
W is selected from the group consisting of —OR⁴, —N(R⁵)₂ and —NHN(R⁵)₂;
R² is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷—S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R³ is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷, —OR⁷, —C(O)OR⁷, —OC(O)R⁷, —C(O)N(R⁶)₂, heterocyclyl and heterocyclylalkyl;
R⁴ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁵ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁶ is selected from the group consisting of hydrogen, alkyl, alkenyl, cycloalkyl, cycloalkylalkyl, aralkyl and aryl; and
R⁷ is selected from the group consisting of hydrogen, alkyl and aralkyl;
Y is selected from the group consisting of an aryl radical of 6 to 10 carbon atoms, 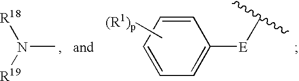
wherein
R¹ is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R¹⁸ is hydrogen or lower alkyl radical;
R¹⁹ is hydrogen, H₂N—, 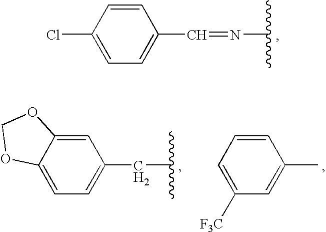
phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy,
m is 0, 1, 2, 3, 4 or 5;
n is 0, 1 or 2;
p is 0, 1, 2, 3, 4 or 5;
q is 0, 1 or 2;
E is selected from the group consisting of 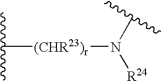
and 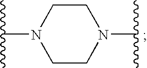
wherein
R²³ is hydrogen or lower alkyl,
R²⁴ is hydrogen or alkyl, and
r is 0, 1, 2 or 3;
as a single stereoisomer, a mixture of stereoisomers, as a racemic mixture of stereoisomers; as a solvate, as a polymorph; or as a pharmaceutically acceptable salt thereof.

2. A method of claim 1 wherein the compound has the formula (1a) 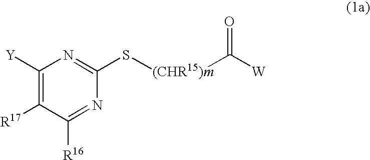
wherein, independently at each occurrence, R¹⁵ and R¹⁷ are each independently selected from the group consisting of hydrogen and lower alkyl radicals;
R¹⁶ is selected from the group consisting of hydrogen, halogen and lower alkoxy radicals;
W is selected from the group consisting of hydroxy, lower alkoxy, —OM and —NHNH₂ radicals, wherein M is selected from the group consisting of alkali metal cation, alkaline earth metal cation and ammonium ion; and
m is 0, 1, 2 or 3.

3. A compound of claim 2 wherein Y is selected from the group consisting of an aryl radical of 6 to 10 carbon atoms, 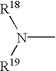
and 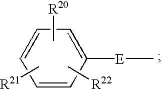
wherein
R²⁰ is selected from the group consisting of a lower alkyl radical, a halo radical, an aryl radical of 6 to 10 carbon atoms and a haloaryl radical of 6 to 10 carbon atoms;
R²¹ is selected from the group consisting of hydrogen, lower alkyl, lower alkoxy and halo radicals; and
R²² is selected from the group consisting of hydrogen and lower alkyl radicals.

4. A method of claim 1 wherein the compound has formula (1b)

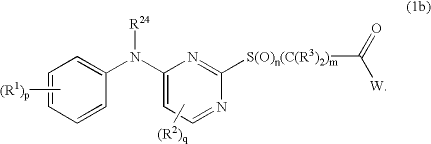

5. A method of claim 4, wherein, independently at each occurrence,
W is selected from the group consisting of —OR⁴ and —N(R⁵)₂;
p is 1, 2, 3 or 4;
q is 1 or 2;
m is 1, 2, 3, 4 or 5; n is 0, 1 or 2;
R¹ has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R² has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2);
R³ has a formula weight of less than 200 and is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷—OR⁷, —C(O)OR⁷, —OC(O)R⁷, and —C(O)N(R⁶)₂;
R⁴ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁵ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁶ has a formula weight of less than 500 and is selected from the group consisting of hydrogen, alkyl, alkenyl, cycloalkyl, cycloalkylalkyl, aralkyl and aryl; and
R⁷ has a formula weight of less than 500 and is selected from the group consisting of hydrogen, alkyl and aralkyl;
as a single stereoisomer, a mixture of stereoisomers, as a racemic mixture of stereoisomers; as a solvate, as a polymorph; or as a pharmaceutically acceptable salt thereof.

6. A method of claim 4 wherein R²⁴ is lower alkyl.

7. A method of claim 1 wherein said cell is a brain cell.

8. A method of claim 1 wherein said β-amyloid is β-amyloid 42.

9. A method of claim 1 wherein β-amyloid production and/or release in the cell is reduced.

10. A method of claim 1 wherein said cell is treated in vitro.

11. A method of treatment comprising modulating the production and/or release of β-amyloid in a non-human mammal in need of said treatment, said method comprising administering to said non-human mammal a compound of the formula (1) 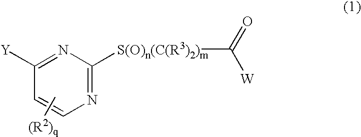
wherein, independently at each occurrence,
W is selected from the group consisting of —OR⁴, —N(R⁵)₂ and —NHN(R⁵)₂;
R² is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R³ is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷, —OR⁷, —C(O)OR⁷, —OC(O)R⁷, —C(O)N(R⁶)₂, heterocyclyl and heterocyclylalkyl;
R⁴ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁵ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁶ is selected from the group consisting of hydrogen, alkyl, alkenyl, cycloalkyl, cycloalkylalkyl, aralkyl and aryl; and
R⁷ is selected from the group consisting of hydrogen, alkyl and aralkyl;
Y is selected from the group consisting of an aryl radical of 6 to 10 carbon atoms, 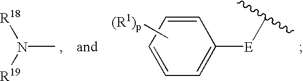
wherein
R¹ is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R¹⁸ is hydrogen or lower alkyl radical;
R¹⁹ is hydrogen, H₂N—, 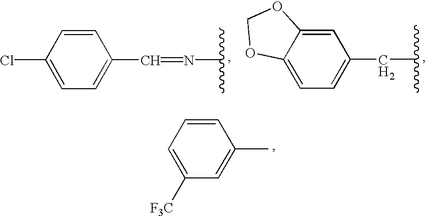
phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy,
m is 0, 1, 2, 3, 4 or 5;
n is 0, 1 or 2;
p is 0, 1, 2, 3, 4 or 5;
q is 0, 1 or 2;
E is selected from the group consisting of 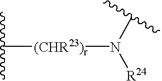
and 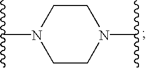
wherein
R²³ is hydrogen or lower alkyl,
R²⁴ is hydrogen or alkyl, and
r is 0, 1, 2 or 3;
as a single stereoisomer, a mixture of stereoisomers, as a racemic mixture of stereoisomers; as a solvate, as a polymorph; or as a pharmaceutically acceptable salt thereof.

12. A method of claim 11 wherein the compound has the formula (1a) 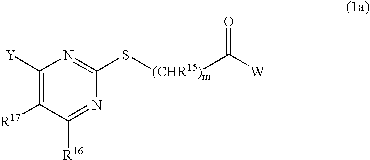
wherein, independently at each occurrence,
R¹⁵ and R¹⁷ are each independently selected from the group consisting of hydrogen and lower alkyl radicals;
R¹⁶ is selected from the group consisting of hydrogen, halogen and lower alkoxy radicals;
R²⁴ is hydrogen or lower alkyl;
W is selected from the group consisting of hydroxy, lower alkoxy, —OM and —NHNH₂ radicals, wherein M is selected from the group consisting of alkali metal cation, alkaline earth metal cation and ammonium ion; and
m is 0, 1, 2 or 3.

13. A compound of claim 12 wherein Y is selected from the group consisting of an aryl radical of 6 to 10 carbon atoms, 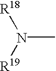
and 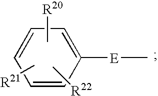
wherein
R²⁰ is selected from the group consisting of a lower alkyl radical, a halo radical, an aryl radical of 6 to 10 carbon atoms and a haloaryl radical of 6 to 10 carbon atoms;
R²¹ is selected from the group consisting of hydrogen, lower alkyl, lower alkoxy and halo radicals; and
R²² is selected from the group consisting of hydrogen and lower alkyl radicals.

14. A method of claim 11 wherein the compound has formula (1b)

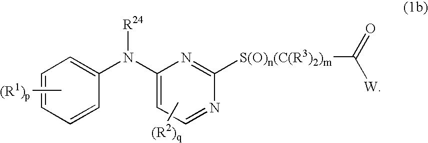

15. A method of claim 14, wherein, independently at each occurrence,
W is selected from the group consisting of —OR⁴ and —N(R⁵)₂;
p is 1, 2, 3 or 4;
q is 1 or 2;
m is 1, 2, 3, 4 or 5; n is 0, 1 or 2;
R¹ has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂—N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R² has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷—C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2);
R³ has a formula weight of less than 200 and is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷, —OR⁷, —C(O)OR⁷, —OC(O)R⁷, and —C(O)N(R⁶)₂;
R⁴ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁵ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁶ has a formula weight of less than 500 and is selected from the group consisting of hydrogen, alkyl, alkenyl, cycloalkyl, cycloalkylalkyl, aralkyl and aryl; and
R⁷ has a formula weight of less than 500 and is selected from the group consisting of hydrogen, alkyl and aralkyl;
as a single stereoisomer, a mixture of stereoisomers, as a racemic mixture of stereoisomers; as a solvate, as a polymorph; or as a pharmaceutically acceptable salt thereof.

16. A method of claim 14 wherein R²⁴ is lower alkyl.

17. A method of claim 11 wherein β-amyloid production and/or release in a brain cell is modulated.

18. A method of claim 11 wherein said β-amyloid is β-amyloid 42.

19. A method of claim 11 wherein said non-human mammal is a mouse, cat, dog or guinea pig.

20. A method of claim 11 wherein β-amyloid production and/or release in a cell is reduced.

21. A method of treatment wherein the production and/or release of β-amyloid is modulated in a human in need of said treatment, said method comprising administering to said human a compound of formula (1) 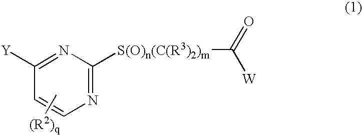
wherein, independently at each occurrence,
W is selected from the group consisting of −OR⁴, —N(R⁵)₂ and —NHN(R⁵)₂;
R² is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶) C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R³ is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷, —OR⁷, —C(O)OR⁷, —OC(O)R⁷, —C(O)N(R⁶)₂, heterocyclyl and heterocyclylalkyl;
R⁴ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁵ is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁶ is selected from the group consisting of hydrogen, alkyl, alkenyl, cycloalkyl, cycloalkylalkyl, aralkyl and aryl; and
R⁷ is selected from the group consisting of hydrogen, alkyl and aralkyl;
Y is selected from the group consisting of an aryl radical of 6 to 10 carbon atoms, 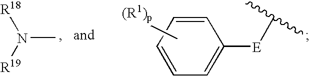
wherein
R¹ is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R¹⁸ is hydrogen or lower alkyl radical;
R¹⁹ is hydrogen, H₂N—, 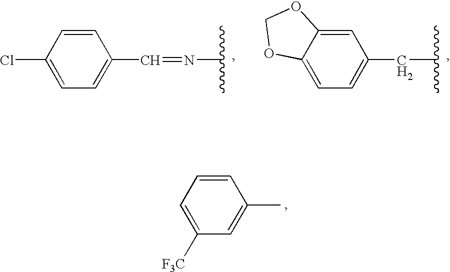
phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy,
m is 0, 1, 2, 3, 4 or 5;
n is 0, 1 or 2;
p is 0, 1, 2, 3, 4 or 5;
q is 0, 1 or 2;
E is selected from the group consisting of 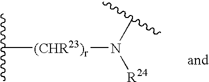
and 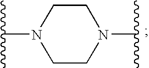
wherein
R²³ is hydrogen or lower alkyl,
R²⁴ is hydrogen or alkyl, and
r is 0, 1, 2 or 3;
as a single stereoisomer, a mixture of stereoisomers, as a racemic mixture of stereoisomers; as a solvate, as a polymorph; or as a pharmaceutically acceptable salt thereof.

22. A method of claim 21 wherein the compound has the formula (1a) 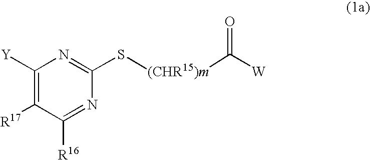
wherein, independently at each occurrence,
R¹⁵ and R¹⁷ are each independently selected from the group consisting of hydrogen and lower alkyl radicals;
R¹⁶ is selected from the group consisting of hydrogen, halogen and lower alkoxy radicals;
R²⁴ is hydrogen or lower alkyl;
W is selected from the group consisting of hydroxy, lower alkoxy, —OM and —NHNH₂ radicals, wherein M is selected from the group consisting of alkali metal cation, alkaline earth metal cation and ammonium ion; and
m is 0, 1, 2 or 3.

23. A compound of claim 22 wherein Y is selected from the group consisting of an aryl radical of 6 to 10 carbon atoms, 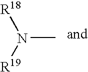
and 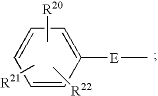
wherein
R²⁰ is selected from the group consisting of a lower alkyl radical, a halo radical, an aryl radical of 6 to 10 carbon atoms and a haloaryl radical of 6 to 10 carbon atoms;
R²¹ is selected from the group consisting of hydrogen, lower alkyl, lower alkoxy and halo radicals; and
R²² is selected from the group consisting of hydrogen and lower alkyl radicals.

24. A method of claim 21 wherein the compound has formula (1b)

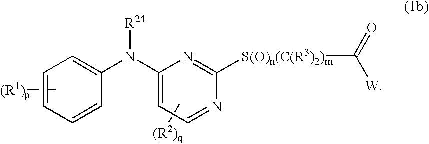

25. A method of claim 24, wherein, independently at each occurrence,
W is selected from the group consisting of —OR⁴ and —N(R⁵)₂;
p is 1, 2, 3 or 4;
q is 1 or 2;
m is 1, 2, 3, 4 or 5; n is 0, 1 or 2;
R¹ has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2), heterocyclyl and heterocyclylalkyl;
R² has a formula weight of less than 500 and is selected from the group consisting of alkyl, alkenyl, alkynyl, heteroalkyl, heteroalkenyl, heteroalkynyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)OR⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —S(O)ₜR⁶ (where t is 0 to 2), —S(O)ₜN(R⁶)₂ (where t is 0 to 2), —OC(S)NR⁶, —NR⁶C(S)OR⁷, —NR⁶S(O)ₜR⁶ (where t is 0 to 2);
R³ has a formula weight of less than 200 and is selected from the group consisting of hydrogen, alkyl, alkenyl, aryl, aralkyl, aralkenyl, cycloalkyl, cycloalkylalkyl, halo, haloalkyl, haloalkoxy, nitro, cyano, —NHOH, —OR⁷, —SR⁷, —C(O)R⁷, —OC(O)R⁷, —C(O)N(R⁶)₂, —C(S)R⁶, —C(O)R⁶, —N(R⁶)₂, —N(R⁶)C(O)R⁶, —N(R⁶)C(O)OR⁷, —OC(S)NR⁶, —NR⁶C(S)OR⁷, —OR⁷, —C(O)OR⁷, —OC(O)R⁷, and —C(O)N(R⁶)₂;
R⁴ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁵ has a formula weight of less than 500 and is selected from the group consisting of hydrogen, alkyl, alkenyl, alkynyl, heteroalkyl, heteroalkenyl, heteroalkynyl, aryl, aralkyl, aralkenyl, cycloalkyl, cycloalkylalkyl, haloalkyl, haloalkoxy, heterocyclyl and heterocyclylalkyl;
R⁶ has a formula weight of less than 500 and is selected from the group consisting of hydrogen, alkyl, alkenyl, cycloalkyl, cycloalkylalkyl, aralkyl and aryl; and
R⁷ has a formula weight of less than 500 and is selected from the group consisting of hydrogen, alkyl and aralkyl;
as a single stereoisomer, a mixture of stereoisomers, as a racemic mixture of stereoisomers; as a solvate, as a polymorph; or as a pharmaceutically acceptable salt thereof.

26. A method of claim 24 wherein R²⁴ is lower alkyl.

27. A method of claim 21 wherein said human is afflicted with Alzheimer's disease.

28. A method of claim 21 wherein said human has suffered a head injury.

29. A method of claim 21 wherein said human has a genetic predisposition or environment exposure that increases the likelihood that said person will develop Alzheimer's disease.

30. A method of claim 21 wherein said human exhibits minimal cognitive impairment suggestive of early stage Alzheimer's disease.

31. A method of claim 21 wherein β-amyloid production and/or release in a brain cell is modulated.

32. A method of claim 21 wherein said β-amyloid is β-amyloid 42.

33. A method of claim 21 wherein P-amyloid production in the human, or β-amyloid release from a cell in the human, is reduced.

34. A method for modulating the production and/or release of β-amyloid in a cell, comprising treating said cell with a compound of the formula (2) 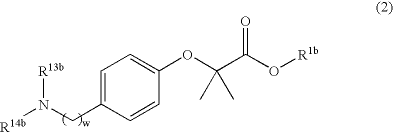
wherein,
R^{1b} is selected from the group consisting of C₁-C₃ alkyl, hydrogen, metal cation and ammonium cation;
R^{13b} and R^{14b} are each independently selected from the group consisting of hydrogen, alkyl, alkenyl, aryl, aralkyl, aralkenyl, cycloalkyl, cycloalkylalkyl, cycloalkylalkenyl, haloalkyl, haloalkenyl, —OR^{12b}, —C(O)OR^{12b}, —N(R^{12b})₂, —C(O)N(R^{12b})₂, —N(R^{12b})C(O)OR^{12b}, heterocyclyl and heterocyclylalkyl;
R^{12b} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and
w is 1, 2 or 3.

35. A method of claim 34 wherein said cell is a brain cell.

36. A method of claim 34 wherein said β-amyloid is β-amyloid 42.

37. A method of claim 34 wherein β-amyloid production and/or release in the cell is reduced.

38. A method of claim 34 wherein said cell is treated in vitro.

39. A method of treatment comprising modulating the production and/or release of β-amyloid in a non-human mammal in need of said treatment, said method comprising administering to said non-human mammal a compound of the formula (2) 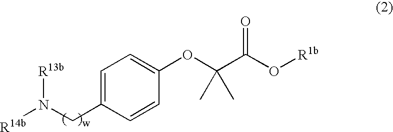
wherein,
R^{1b} is selected from the group consisting of C₁-C₃ alkyl, hydrogen, metal cation and ammonium cation;
R^{13b} and R^{14b} are each independently selected from the group consisting of hydrogen, alkyl, alkenyl, aryl, aralkyl, aralkenyl, cycloalkyl, cycloalkylalkyl, cycloalkylalkenyl, haloalkyl, haloalkenyl, —OR^{12b}, —C(O)OR^{12b}, —N(R^{12b})₂, —C(O)N(R^{12b})₂, —N(R^{12b})C(O)OR^{2b}, heterocyclyl and heterocyclylalkyl;
R^{12b} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and
w is 1, 2 or 3.

40. A method of claim 39 wherein β-amyloid production and/or release in a brain cell is modulated.

41. A method of claim 39 wherein said β-amyloid is β-amyloid 42.

42. A method of claim 39 wherein said non-human mammal is a mouse, cat, dog or guinea pig.

43. A method of claim 39 wherein β-amyloid production and/or release in a cell is reduced.

44. A method of treatment comprising modulating the production and/or release of β-amyloid in a human in need of said treatment, said method comprising administering to said human a compound of the formula (2) 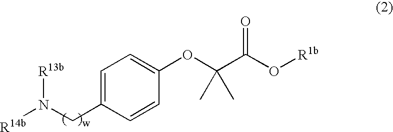
wherein,
R^{1b} is selected from the group consisting of C₁-C₃ alkyl, hydrogen, metal cation and ammonium cation;
R^{13b} and R^{14b} are each independently selected from the group consisting of hydrogen, alkyl, alkenyl, aryl, aralkyl, aralkenyl, cycloalkyl, cycloalkylalkyl, cycloalkylalkenyl, haloalkyl, haloalkenyl, —OR^{12b}, —C(O)OR^{12b}, —N(R^{12b})₂, —C(O)N(R^{12b})₂, —N(R^{12b})C(O)OR^{2b}, heterocyclyl and heterocyclylalkyl;
R^{12b} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and
w is 1, 2 or 3.

45. A method of claim 44 wherein said human is afflicted with Alzheimer's disease.

46. A method of claim 44 wherein said human has suffered a head injury.

47. A method of claim 44 wherein said human has a genetic predisposition or environment exposure that increases the likelihood that said person will develop Alzheimer's disease.

48. A method of claim 44 wherein said human exhibits minimal cognitive impairment suggestive of early stage Alzheimer's disease.

49. A method of claim 44 wherein β-amyloid production and/or release in a brain cell is modulated.

50. A method of claim 44 wherein said β-amyloid is β-amyloid 42.

51. A method of claim 44 wherein β-amyloid production in the human, or β-amyloid release from a cell in the human, is reduced.

52. A compound of the formula (1c) 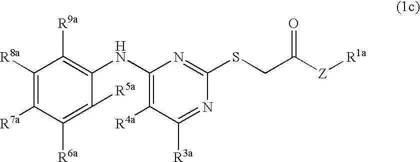
wherein, independently at each occurrence,
R^{1a} is an organic moiety having at least 4 carbons;
Z is selected from —O—, —NH—NH—, and —N(R^{2a})—;
R^{2a} is selected from hydrogen and C₁-C₃₀ organic moieties with the proviso that R^{1a} and R^{2a} can join together with the nitrogen to which they are both attached and form a heterocyclic moiety;
R^{3a} and R^{4a} are each independently selected from the group consisting of hydrogen, halogen, lower alkyl and lower alkoxy radicals;
R^{5a}, R^{6a}, R^{7a}, R^{8a} and R^{9a} are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R^{10a}—N═N—O—R^{11a}, —OR^{12a}, —C(O)OR^{12a}, —N(R^{12a})₂, —C(O)N(R^{12a})₂, —N(R^{12a})C(O)OR^{11a}, heterocyclyl and heterocyclylalkyl;
R^{10a} is a bond or a straight or branched alkylene or alkenylene chain;
R^{11a} is hydrogen, alkyl or aralkyl; and
R^{12a} is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
with the proviso that Z is not NR^{2a} when R^{3a} is Cl, R^{4a} is H, R^{5a} is H, R^{6a} is H, R^{7a} is H, R^{8a} is methyl and R^{9a} is methyl.

53. A compound of claim 52 wherein Z is —O— and R^{1a} is an organic group having less than 30 carbons and a formula weight of less than 1,000.

54. A compound of claim 52 wherein Z is —N(H)— and R^{1a} is an organic group having less than 30 carbons and a formula weight of less than 1,000.

55. A compound of claim 52 wherein Z is —N(R²)— and R^{1a} is an organic group having less than 30 carbons and a formula weight of less than 1,000.

56. A compound of claim 52 wherein R^{1a} is selected from the group consisting of alkyl, alkenyl, aryl, aralkyl, aralkenyl, cycloalkyl, cycloalkylalkyl, cycloalkylalkenyl, haloalkyl, haloalkenyl, —OR^{12a}, —C(O)OR^{12a}, —N(R^{12a})₂, —C(O)N(R^{12a})₂, —N(R^{12a})C(O)OR^{11a}, heterocyclyl and heterocyclylalkyl.

57. A compound of claim 52 wherein R^{1a} is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4.

58. A compound of claim 52 wherein R^{1a} is a fragment of insulin wherein said insulin fragment binds to an insulin receptor.

59. A compound of claim 58 wherein said fragment of insulin consists of:
(a) a peptide chain having 14 to 21 amino acid residues from the N-terminus of insulin chain A; and
(b) another peptide chain having 16 to 22 amino acid residues from the N-terminus of insulin chain B.

60. A compound of claim 52 wherein R^{1a} is a protein that binds to a transferrin receptor.

61. A compound of claim 52 wherein R^{1a} is an antibody or a fragment thereof capable of binding to a ligand in the brain.

62. A compound of claim 52 wherein said antibody is a monoclonal antibody.

63. A compound of claim 52 wherein R^{1a} is a growth factor.

64. A compound of claim 63 wherein said growth factor is EGF.

65. A compound of claim 52 wherein each of R^{5a}, R^{6a}, R^{7a}, R^{8a} and R^{9a} is independently selected from the group consisting of hydrogen, halogen, lower alkyl and lower alkoxy radicals.

66. A compound of claim 52 having enhanced penetration of the blood brain barrier relative to the corresponding compound wherein R^{1a} is hydrogen when Z is —O—, and both R^{1a} and R^{2a} are hydrogen when Z is —N(R^{2a})—.

67. A composition comprising a compound of claim 52 and a pharmaceutically acceptable carrier, diluent or excipient.

68. A compound of the formula (1d) 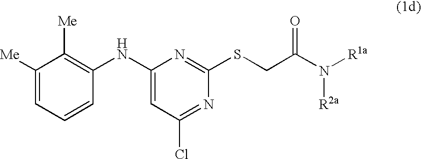
wherein,
R^{1a} is a hydrophobic moiety selected from non-aromatic organic moieties having at least 10 carbon atoms and aromatic moieties having at least 6 carbons, and R^{2a} is hydrogen; or
each of R^{1a} and R^{2a} is selected from hydrophobic organic moieties having at least one carbon atom, with the proviso that R^{1a} and R^{2a} in total have at least six carbon atoms, and with the further proviso that R^{1a} and R^{2a} can join together with the nitrogen to which they are both bonded and form a heterocyclic moiety.

69. A composition comprising a compound of claim 68 and a pharmaceutically acceptable carrier, diluent or excipient.

70. A compound that (1) is a PPARα agonist and/or a PPARδ agonist, and (2) regulates the production and/or release of β-amyloid in cells.

71. A composition comprising a compound of claim 70 and a pharmaceutically acceptable carrier, diluent or excipient.
